(19) United States
(12) Patent Application Publication
(10) Pub. No.: US 2003/0119792 A1
(43) Pub. Date: Jun. 26, 2003

(54) Method of inhibiting the production and/or effects of intestinal pro-inflammatory cytokines, prostaglandins and others

(76) Inventor: Manuel Merlos Roca, Barcelona (ES)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/259,868

(22) Filed: Sep. 30, 2002

Related U.S. Application Data
(60) Provisional application No. 60/325,193, filed on Sep. 28, 2001.

(51) Int. Cl. A61K 31/655; A61K 31/4745

(52) U.S. Cl. 514/150; 514/303

(57) ABSTRACT

The present invention shows that UR-12746-S, a novel locally acting compound which combines 5-ASA (an anti-inflammatory) and UR-12715 (a PAF antagonist) through an azo link, and analogous azo derivatives of 5-aminosalicylic acid compounds are able to inhibit cytokine production (IL-8, IL-1β and TNF-α) in vitro, and are shown to have intestinal anti-inflammatory activity in vivo. Moreover, daily oral administration of azo derivatives of 5-aminosalicylic acid sodium salts are able to alleviate and/or prevent relapse of inflammatory disease induced in colitic rats. This beneficial effect is evidenced by a significant reduction in colonic myeloperoxidase activity and by a significant decrease in colonic IL-1β and TNF-α.

### CROSS REFERENCE TO RELATED APPLICATIONS

This application claims priority to U.S. Provisional Application 60/325,193, filed Sep. 28, 2001, the disclosure of which is hereby incorporated in its entirety by reference.

### FIELD OF THE INVENTION

The present invention relates to methods of preparing azo derivatives of 5-aminosalicylic acid, to pharmaceutical compositions containing these compounds, and to their use in the treatment or prevention of relapse of inflammatory bowel disease.

### BACKGROUND OF THE INVENTION

Chronic inflammatory bowel disease (IBD), mainly ulcerative colitis and Crohn's disease, is a naturally remitting and recurring condition of the digestive tract probably related to an abnormal exacerbated immune response to an otherwise innocuous stimulus which is not properly abrogated by the feedback system that normally down-regulates the mucosal tissue response to luminal factors (Fiocchi, 1998). As a consequence, several types of cells, particularly immune system cells, including lymphocytes, neutrophils, macrophages, and mast cells, are attracted to the site of initial injury and infiltrate the tissues there. This results in an overproduction of a variety of proinflammatory mediators such as eicosanoids, platelet activating factor (PAF), reactive oxygen metabolites and cytokines, thus influencing mucosal integrity and leading to excessive tissue injury (Katz et al., 1999; Podolsky & Fiocchi, 2000). Moreover, most of these mediators induce the biosynthesis and release of other mediators, generating a “vicious cycle” that may result in the unnecessary propagation and perpetuation of the inflammatory response. At present, a specific causal treatment of IBD is still not available and, for this reason, the best chance to effectively counteract the exacerbated immune response that characterises IBD may be to interfere with multiple stages of the inflammatory cascade, preferably with a unique drug treatment (Kho et al., 2001).

The pharmacological profile of (Z)-2-hydroxy-5-[[4-[3-[4-[(2-methyl-1H-imidazo[4,5-c]pyridin-1-yl)methyl]-1-piperidinyl]-3-oxo-1-phenyl-1-propenyl]phenyl]azo] benzoic acid and its sodium salt (UR-12746 and UR-12746-S) makes it a good candidate to be developed for the treatment of IBD. UR-12746 combines, through an azo bond, two active molecules for the treatment of these intestinal conditions: 5-aminosalicylic acid (5-ASA) and UR-12715, the latter being a compound which displays PAF antagonist activity.

In fact, in a previous study, the intestinal anti-inflammatory activity of UR-12746 (free acid) in the TNBS experimental model of rat colitis was demonstrated (Galvez et al., Brit. J. Pharm. v.130:1949-1959 (2000), the disclosure of which is incorporated herein by reference). This anti-inflammatory effect is probably related to the additive effects locally exerted by both compounds once they are released in the intestinal lumen following reduction of the azo bond by intestinal bacteria. 5-ASA derivatives have been previously described to display intestinal anti-inflammatory activity through a combination of different mechanisms, including antioxidant and/or radical scavenging properties, inhibition of leukocyte chemotaxis, and downregulation of the synthesis and/or release of proinflammatory cytokines and eicosanoids (Travis & Jewell, 1994a; Makins & Cowan, 2001). Blockade of the PAF receptor by specific antagonists has been shown to have a beneficial effect in experimental models of intestinal inflammation as well (Wallace, 1988; Meenan et al., 1996).

Galvez et al. (Galvez) and U.S. Pat. No. 5,747,477 (the ′477 patent) disclose, respectively, uses of UR-12746 free acid and uses of generic compounds of Formula I, see below, for the treatment of IBD. In Galvez, UR-12746 free acid is shown to inhibit Platelet Activating Factor (PAF) activity, reduce colonic myeloperoxidase (MPO) activity, and reduce IL-1β production at dosages of 50-100 mg/kg in the TNBS-induced rat colitis model over a 4 week time span, and shown to reduce LTB₄ production but only at a dosage of 100 mg/kg and only after 3 and 4 weeks of administration. No assessment of the effects of UR-121746 on colitis relapse are performed. A significant reduction in colonic damage is only seen at the 100 mg/kg dosage. The ′477 patent discloses that, in the same model, compounds of Formula I administered at 100 mg/kg over a 7 day period (with no relapse simulation) reduced colonic damage, reduced PAF activity, and also reduced the colonic production of LTB₄ and PGE₂.

However, the methods of the present invention provide significant advances over either of these references.

### SUMMARY OF THE INVENTION

The present study demonstrates the effects of certain azo derivatives of 5-aminosalicylic acid, UR-12746-S (salt or free acid), on the production and release of several proinflammatory cytokines by intestinal cells both in vitro and in vivo. In the in vitro studies, three different cell lines have been used: HT-29 cells, as a model of intestinal epithelium; and U-937 and THP-1 as models of monocyte/macrophage cells, representing different stages of development--U937 represents a relative immature stage of monocyte lineage while THP1 represents an advanced stage of myelomonocytic development. The three cytokines studied, interleukin-8 (IL-8), interleukin-1β (IL-1β) and tumour necrosis factor-α (TNF-(α), have been shown to play a key role in the pathogenesis of IBD (Katz et al., 1999).

We have also shown the effectiveness of the administration of the azo derivatives of 5-aminosalicylic acid of Formula I in an in vivo model for the relapse of inflammatory disease. The beneficial effects both in the prevention and amelioration or the negative effects of a relapse are demonstrated. The experimental model involves the reactivation of colitis. Reactivation is achieved by a second administration of the hapten trinitrobenzenesulphonic acid (TNBS) once the initial injury from the experimental colitis is in the process of recovery.

Accordingly, the present invention provides for a method of ameliorating negative effects of relapse of inflammatory bowel disease in a mammal comprising administering to a mammal having suffered from inflammatory bowel disease an effective amount of a compound of Formula I:

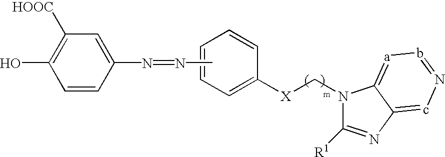

wherein: the 4-hydroxy-3-carboxyphenylazo moiety can be at the 3- or 4-position of the benzene ring; m represents 1 or 2; R¹ represents C₁₋₄ alkyl or C₃₋₇ cycloalkyl; a, b and c represent CR², wherein each R² independently represents hydrogen or C₁₋₄ alkyl; X represents a group of formula (i) or (ii):

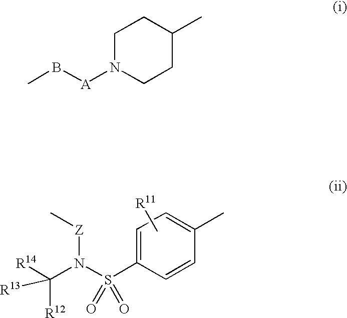

wherein these groups are bound to the phenyl ring in formula I via B and Z, respectively; A represents -CO-, -SO₂-, -NHCO- or -OCO-; B represents a group of formula (iii), and when A represents -CO- or -SO₂-, B can also represent a group of formula (iv), (v), (vi) or (vii);

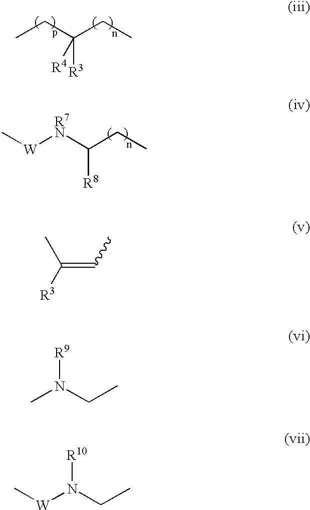

n represents 0, 1, 2 or 3; p represents O or I; R³represents hydrogen, C₁₋₄ alkyl, C₁₋₄ haloalkyl, C₃₋₇ cycloalkyl, C₁₋₄ alkoxy-C₁₋₄ alkyl or aryl; R⁴ represents hydrogen, C₁₋₄ alkyl, -COOR⁵ or -CONR5R⁶, and when A represents -CO- or -SQ-, then R⁴ can also represent -NR⁵R⁶, -NR⁷C(=O)OR⁵, -NR⁷C(=O)R⁵, -NR⁷C(=O)NR⁵R⁶ or -NR⁷SO₂R⁵; or R³ and R⁴ together form a C₂₋₆polymethylene chain; R⁵represents C₁₋₄ alkyl, aryl or aryl-C₁₋₄ alkyl; R⁶ and R⁷ independently represent hydrogen or C₁₋₄ alkyl; W represents -OC(=O)-, -C(=O)-, -NR⁶C(=O)- or -SO₂-; R⁸ represents aryl; R9 represents C₁₋₄ alkyl, C₃₋₇ cycloalkyl, -C(=O)OR⁵, -C(=O)R⁵, -C(=O)NR⁵R⁶, or-SO2R⁵; R¹⁰ represents C₁₋₄ alkyl, C₃₋₇ cycloalkyl, aryl, or aryl-C₁₋₄ alkyl; Z represents (CH₂)_{q}CO or -(CH₂)ᵣ-q represents 0, 1 or 2; r represents 1 or 2; R¹¹ represents hydrogen or halogen; R¹² and R¹³ independently represent hydrogen, C₆ alkyl, C ₃₋₇cycloalkyl or C₃₋₇ cycloalkyl-C₁₋₆ alkyl; or R¹² and R¹³ together form a C₂₋₆ polymethylene chain; R¹⁴ represents -COR¹⁵, -COOH, -COOR¹⁵, -CONR¹⁶R¹⁷, -C₁₋₆ alkyl-OR¹⁵, -C₁₋₆ alkyl-OC(=O)R¹⁵ or -C₁₋₆ alkyl-OC(=O)NR¹⁶R¹⁷ R¹⁵ represents C₁₋₆ alkyl, C₂₋₆ alkenyl, C₂₆alkynyl, C₃₋₇ cycloalkyl or C₁₋₆ haloalkyl; R¹⁶ and R¹⁷ independently represent hydrogen or any of the meanings disclosed for R¹⁵;

aryl, whenever appearing in the above definitions, represents phenyl or phenyl substituted with 1, 2, 3 or 4 groups independently selected from halogen, C₁₋₄alkyl, C₁₋₄alkoxy, hydroxy, C₁₋₄ haloalkyl, C₁₋₄ haloalkoxy, C₁₋₄ alkylcarbonyl, C₁₋₄ alkylcarbonyloxy, C₁₋₄ alkoxycarbonyl, C₁₋₄ alkylsulfonyl, C₁₋₄ alkylsulfinyl, C₁₋₄ alkylthio, or C₁₋₄ alkylcarbonylamino; including their pharmaceutically acceptable salts and solvates.

The present invention also provides a method of preventing a relapse of inflammatory bowel disease in a mammal wherein effective amounts of compounds of Formula I are administered to a mammal having suffered from inflammatory bowel disease.

In particular, certain compounds of Formula I are found to be particularly useful in the methods of the present invention, and include those listed below as Group I:

1-[[1-[3-[4-(4-hydroxy-3-carboxyphenylazo) phenyl]-3methylbutanoyl]-4-piperidyl] methyl]-1H-2-methylimidazole [4,5-c] pyridine;

trans- 1-[[1-[3-[4-(4-hydroxy-3-carboxyphenylazo) phenyl]-3-phenylpropenoyl]-4-piperidyl] methyl]-1H-2-methylimidazole [4,5-c] pyridine;

1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c] pyridine

[N-[4-(4-hydroxy-3-carboxyphenylazo) benzoyl]-N-[4-(1 H-2-methylimidazole [4,5-c]pyridylmethyl) phenylsulfonyl]-L-leucine ethyl ester;

[N-[4-(4-hydroxy-3-carboxyphenylazo) benzyl]-N-[4-(1 H-2-methylimidazole [4,5-c]pyridylmethyl) phenylsulfonyl]-L-leucine ethyl ester;

[N-[4-(4-hydroxy-3-carboxyphenylazo) benzyl]-N-[(S)- 1-isobutyl-ethoxyethyl]-N-[4-(1H-2-methylimidazole [4,5-c] pyridylmethyl) phenylsulfonamide;

cis- 1-[[1-[3-[4-(4-hydroxy-3-carboxyphenylazo) phenyl]-3-phenylpropenoyl]-4-piperidyl] methyl]-1H-2-methylimidazole [4,5-c] pyridine; and

(Z)-2-hydroxy-5-[[4-[3-[4-[(2-methyl-1H-imidazo[4,5-c] pyridin-1-yl)methyl]- 1-piperidinyl]-3-oxo-1-phenyl-1-propenyl]phenyl]azo] benzoic acid.

Preferably, the pharmaceutically acceptable salts of these compounds are used in the inventive methods, preferably the alkali or alkaline-earth metal salts thereof, such as sodium, potassium, calcium, magnesium, aluminum, lithium, zinc, and the like; or their acid addition salts, such as hydrochloric acid, hydrobromic acid, hydroiodic acid, phosphoric acid, and the like; or salts of amines, such as ammonia, aklyamines, hroxyalkylamines, lysine, arginine, glutamine, and the like.

The methods of the present invention employing compounds of Formula I, and particularly those listed in Group I, and their pharmaceutically acceptable salts and solvates, at doses of 10-10,000 mg, preferably about 25 mg/kg to about 100 mg/kg, including dosages of at least 100 mg/kg, 100 mg/kg, at least 50 mg/kg, 50 mg/kg, less than 50 mg/kg, or about 25 mg/kg, daily, in divided doses daily, and including doses administered for periods of time including 7 days, one week, two weeks, four weeks, and at least four weeks, are shown to decrease colonic damage or deterioration, promote mucosal healing, prevent relapse of colitis, inhibit the activities of PAF, NOS, and MPO, and reduce colonic production of cytokines, eicosanoids, and prostaglandins, namely LTB₄, TNF-α, IL-1β, IL-8, and PGE₂, all at dosages significantly lower than those previously disclosed. Furthermore, the methods of the present invention are effective to reduce the production and activity of transcription factors in colonic cells, as exemplified by NF-KB.

The methods of the present invention are particularly effective in treatment of inflammatory bowel disease, including chronic gastrointestinal inflammation, colitis, chronic colitis, ulcerative colitis or Crohn's disease. The methods of the present invention are most effective when the compounds of Formula I, and particularly those listed in Group I, and their pharmaceutically acceptable salts and solvates, are delivered orally or rectally and optionally in combination with pharmaceutically acceptable carriers, excipients, and the like.

In yet another embodiment of the methods of the present invention, there is provided a method of inhibiting production of one or more cytokines in a mammal, comprising administering to a mammal in need thereof an effective amount of a compound of Formula I or a compound selected from Group I, and the pharmaceutically acceptable salts and solvates thereof. The methods of this embodiment are particularly effective wherein one or more of the cytokines includes interleukin-8 or tumor necrosis factor-α. The methods of these embodiments may be practiced according to any one or more, or any combination of, the procedures and protocols of the methods described above.

A further embodiment of the present invention comprises a method of inhibiting cellular production of one or more of prostaglandins or proinflammatory mediators comprising contacting cells with an effective amount of a compound of Formula I or a compound selected from Group I, and the pharmaceutically acceptable salts and solvates thereof. Included within this embodiment are methods wherein said prostaglandins or proinflammatory mediators comprise prostaglandin E₂, wherein said prostaglandins or proinflammatory mediators comprise eicosanoids, wherein said prostaglandins or proinflammatory mediators comprise reactive oxygen metabolites, wherein said prostaglandins or proinflammatory mediators comprise platelet activating factor, wherein said prostaglandins or proinflammatory mediators comprise cytokines, wherein said cytokines comprise one or more of interleukin-8, interleukin 1-β, or tumor necrosis factor-α or wherein said prostaglandins or proinflammatory mediators comprise colonic myeloperoxidase (MPO), colonic leukotriene B₄ (LTB₄), colonic tumor necrosis factor-alpha (TNF-α), colonic interleukin 8 (IL-8), colonic nuclear factor kappa B (NF-KB), colonic prostaglandins E₂ (PGE₂), colonic nitric oxide synthase (NOS), plasmatic nitrites and nitrates or any combination thereof. The methods of this embodiment, and those listed below, may be practiced according to the procedures and protocols of the methods described above, or may include the methods wherein the effective amount is within the range of about 25 mg/kg up to but not including 50 mg/kg, is at least 50 mg/kg, or is within the range of about 10⁻⁴ molar to 10⁻⁶ molar plasma or intestinal lumen concentration and wherein said cells are contacted with said effective amount of said compound for at least 30 minutes, at least 30 minutes daily, and at least 30 minutes daily for periods of 7 days, 2 weeks, 4 weeks, or at least 4 weeks.

An even further embodiment of the present invention is a method of inhibiting mucosal tissue deterioration comprising contacting mucosal tissue with an effective amount of a compound of Formula I or a compound selected from Group I, and the pharmaceutically acceptable salts and solvates thereof.

Another embodiment of the inventive method is a method of inhibiting mucosal inflammation comprising contacting mucosal tissue with an effective amount an effective amount of a compound of Formula I or a compound selected from Group I, and the pharmaceutically acceptable salts and solvates thereof

In yet an even further embodiment of the present inventive, a method is provided for inhibiting tissue infiltration by immune system cells, comprising contacting said mucosal tissue with an effective amount of a compound of Formula I or a compound selected from Group I, and the pharmaceutically acceptable salts and solvates thereof. The method is of particular use wherein the infiltration is associated with production of myeloperoxidase by the tissues being infiltrated.

Another embodiment of the inventive method is a method of inhibiting effects of platelet activating factor on tissues comprising contacting the tissues with an effective amount an effective amount of a compound of Formula I or a compound selected from Group I, and the pharmaceutically acceptable salts and solvates thereof.

Hence, the use of compounds of Formula I, and particularly of those selected from Group I, and the pharmaceutically acceptable salts and solvates thereof, under methods of the present invention represents an important advance in the field.

### BRIEF DESCRIPTION OF THE FIGURES

FIG. 1. Effect of UR-12715, 5-ASA and the combination UR-12715 plus 5-ASA on IL-8 production in HT-29 cells. **P<0.01 vs. UR-12715+5-ASA.

FIG. 2. Effect of UR-12715, 5-ASA and the combination UR-12715 plus 5-ASA on IL-1β production in THP-1 cells. # P<0.05, ## P<0.01 vs. 5-ASA; * P<0.05,** P<0.01 vs. UR-12715+5-ASA.

FIG. 3. Effect of UR-12715, 5-ASA and the combination UR-12715 plus 5-ASA on TNFα production in U937 cells. # P<0.05, ## P<O.0L vs. 5-ASA.

FIG. 4. Effects of UR-12746-S (25 and 50 mg kg⁻¹) on colonic myeloperoxidase (MPO) activity in reactivated TNBS colitis. Data are expressed as mean ± s. e. mean.** P<0.01 vs. TNBS control group; # P<0.05, ## P<0.01 vs. non colitic group.

FIG. 5. Effects of UR-12746-S (25 and 50 mg kg⁻¹) on colonic IL-1, levels in reactivated TNBS colitis. Data are expressed as mean ± s. e. mean. *P<0.05,** P<0.01 vs. TNBS control group; ## 1 P<0.01 vs. non colitic group.

FIG. 6. Effects of UR-12746-S (25 and 50 mg kg⁻¹) on colonic TNF-α levels in reactivated TNBS colitis. Data are expressed as mean ± s. e. mean. P<0.05, P<0.01 vs. TNBS control group; # P<0.05, ## P<0.01 vs. non colitic group.

FIG. 7. Effects of UR-12746-S in U-937 cells. UR-12746-S appears to be more potent (shifts the dose response curve to the left) in inhibiting the production of TNF-α in U-937 cells when compared to the individual components, a combination of the components and sulfasalazine.

FIG. 8. Effects of UR-12746-S in the production of TNF-α in mononuclear cells. UR-12746-S appears to inhibit the production of TNF-α in mononuclear cells to a greater extent than either UR-12715 or a combination of UR-12715 and 5-ASA at a concentration of 10 uM.

### DETAILED DESCRIPTION OF THE PREFERRED EMBODIMENTS

The methods of the present invention, employing compounds of Formula I at doses of 10-10,000 mg, preferably about 25 mg/kg to about 100 mg/kg, including dosages of at least 100 mg/kg, 100 mg/kg, at least 50 mg/kg, 50 mg/kg, less than 50 mg/kg, or about 25 mg/kg, daily, in a single dose or optimally in two or more divided doses, and including doses administered for periods of time including 7 days, one week, two weeks, four weeks, and at least four weeks, are shown to decrease colonic damage or deterioration, promote mucosal healing, prevent relapse of colitis, inhibit the activities of PAF, NOS, and MPO, and reduce colonic production of cytokines, eicosanoids, and prostaglandins, namely LTB₄, TNF-α, IL-1β, IL-8, and PGE₂, all at dosages significantly lower than those previously disclosed. Furthermore, methods of the present invention are effective to reduce the production and activity of transcription factors in colonic cells, as exemplified by NF-KB.

The aim of this study is to evaluate the effect of compounds used under the methods of the present invention in a model of ulcerative colitis (UC) and to study the mechanisms of action involved in their intestinal anti-inflammatory activity. After oral administration, azo derivatives of 5-aminosalicylic acid of Formula I are cleaved by colonic bacterial azoreductase, delivering a platelet activating factor (PAF) antagonist, for example UR-12715, and 5-aminosalicylic acid (5-ASA), which displays intestinal anti-inflammatory activity. The HT-29, U937 and THP-1 cell lines are used as in vitro model. As an in vivo model, colitis is induced in rats by TNBS, and the relapse occurring in chronic colitis is simulated by a second administration of the same TNBS compound 2 weeks or 4 weeks after the initial dose.

We find that administration of compounds of Formula I, particularly those selected from Group I, and even more particularly UR-12746-S, are effective to prevent relapse in the experimental model of chronic inflammatory bowel disease, and to ameliorate the negative effects of relapse in cases in which relapse occurs. In the experimental model, the colonic segments appear ulcerated and inflamed, with a concomitant increase in the colonic weight/length ratio, one and two weeks after colitis induction by administration of TNBS in control rats untreated by methods of the present invention.

However, following treatment by methods of the present invention, decreased colonic damage and deterioration, promoted mucosal healing, decreased neutrophil tissue infiltration, and prevented relapse of colitis are shown. Administration of UR-12746-S is able to attenuate the impact of the reactivation in the inflammatory process, as shown macroscopically on colonic macroscopic damage scores and on colonic weight/length ratios, which show no statistical differences when comparing animals treated by methods of the present invention with control colitic animals, which were not administered a second TNBS dose to induce relapse. Furthermore, following treatment with UR-12746-S, there is a decrease in colonic MPO activity which, since MPO activity is considered as a marker of neutrophil infiltration into the inflamed mucosa, represents a significant anti-inflammatory response in vivo. This shows the correlation between the intestinal anti-inflammatory effect and the lower leukocyte infiltration in the inflamed mucosa also evidenced by macroscopic inspection. In sum, these data - particularly the macroscopic data - indicate that the methods of the present invention are effective to prevent relapse of IBD or to ameliorate the severity of negative effects of relapse, if relapse occurs.

Further, we find that the individual breakdown products, UR-12715 and 5-ASA, are able to inhibit the IL-8 increase produced by LPS in HT-29 cells. The maximum inhibition is reached at the concentration of 10⁻⁶ M (30-40%), and this inhibition is significantly higher when both compounds are administered together at 10⁻⁴ M (60%), as they would be in the intestinal lumen following azoreduction of UR-12746-S. THP-1 and U937 cells treated by PMA and LPS show an increase in IL-1β and TNF-α, respectively. Production of both of these cytokines is inhibited by UR- 12715 in a concentration-dependent manner. However 5-ASA has only a weak effect inhibiting IL-1β production. Results obtained in vivo show that UR-12746-S (25 and 50 mg kg⁻¹), for example, reduces colitic damage as well as the activity of the enzyme myeloperoxidase (MPO), a promoter of neutrophil tissue infiltration, during the first two weeks of colitis and after colitic relapse.

We find that UR-12746-S administration is able to ameliorate the TNBS-induced increase of IL-1β production two weeks after the beginning of the experiment without having any effect on TNF-α increase at that time. However, UR-12746-S decreases the production of both cytokines induced by a second administration of TNBS one week following the initial administration, and prevents their production to such an extent as to prevent the symptoms of relapse when the methods of the present invention are applied for four weeks prior to the second administration of TNBS, following an initial administration. Taken together, these results show that the new compound UR-12746-S is an effective drug for treatment of chronic colitis, that it ameliorates the negative consequences of colitic relapse, and that it prevents relapse in some cases - in other words, it promotes and maintains a state of remission in the chronic colitic. This compound is able to inhibit the inflammatory reaction generated by PAF and to break down the vicious cycle generated by cytokine production, and prevent relapse of IBD. The combination of PAF-R inhibition elicited by UR-12715 and the intestinal anti-inflammatory action of 5-ASA produce an additive beneficial effect in the experimental colitic when delivered as the conjugated compound UR-12746-S by the methods of the present invention

Compounds used in the methods of the present invention are described in Formula I:

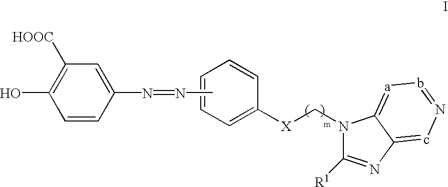

wherein: the 4-hydroxy-3-carboxyphenylazo moiety can be at the 3- or 4-position of the benzene ring; m represents 1 or 2; R¹ represents C₁₋₄ alkyl or C₃₋₇ cycloalkyl; a, b and c represent CR², wherein each R independently represents hydrogen or C₁₋₄alkyl; X represents a group of formula (i) or (ii):

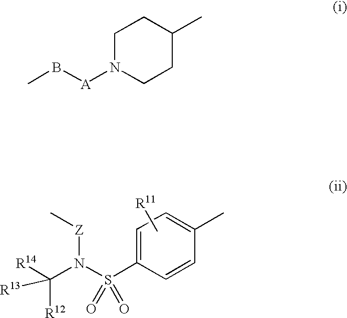

wherein these groups are bound to the phenyl ring in formula I via B and Z, respectively; A represents -CO-, -SO₂-, -NHCO- or -OCO-; B represents a group of formula (iii), and when A represents -CO- or -SO₂-, B can also represent a group of formula (iv), (v), (vi) or (vii);

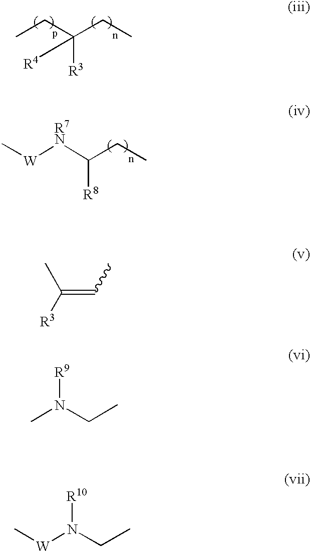

n represents 0, 1, 2 or 3; p represents O or I; R³represents hydrogen, C₁₋₄ alkyl, C₁₋₄ haloalkyl, C₃₋₇ cycloalkyl, C₁₋₄alkoxy-C₁₋₄ alkyl or aryl; R⁴ represents hydrogen, C₁₋₄ alkyl, -COOR⁵ or -CONR⁵R ⁶, and when A represents -CO- or -SQ-, then R⁴ can also represent -NR⁵R⁶, -NR⁷C(=O)OR⁵, -NR⁷C(=O)R⁵, -NR⁷C(=O)NR⁵R⁶ or -NR⁷SO₂R⁵; or R³ and R⁴ together form a C₂₋₆ polyrnethylene chain; R⁵ represents C₁₋₄ alkyl, aryl or aryl-C₁₋₄ alkyl; R⁶ and R⁷ independently represent hydrogen or C₁₋₄ alkyl; W represents -OC(=O)-, -C(=O)-, -NR⁶C(=O)- or -SO₂-; R⁸ represents aryl; R9 represents C₁₋₄ alkyl, C₃₋₇ cycloalkyl, -C(=O)OR⁵, -C(=O)R⁵, -C(=O)NR⁵R⁶, or-SO₂R⁵; R¹⁰ represents C₁₋₄ alkyl, C₃₋₇ cycloalkyl, aryl, or aryl-C₁₋₄ alkyl; Z represents (CH₂)_{q}CO- or CH₂)ᵣ-q represents 0, 1 or 2; r represents 1 or 2; R¹¹ represents hydrogen or halogen; R¹² and R¹³ independently represent hydrogen, C₁₋₆ alkyl, C₃₋₇ cycloalkyl or C₃₋₇ cycloalkyl-C₁₋₆ alkyl; or R¹² and R¹³ together forn a C₂₋₆ polymethylene chain; R¹⁴ represents -COR¹⁵-COOH, -COOR¹⁵, -CONR¹⁶R¹⁷, -C₁₋₆ alkyl-OR¹⁵, -C₁₋₆alkyl-OC(=O)R¹⁵ or -C₁₋₆ alkyl-OC(=O)NR¹⁶R¹⁷; R¹⁵ represents C₁₋₆ alkyl, C₂₋₆ alkenyl, C₂₆alkynyl, C₃₋₇cycloalkyl or C₁₋₆ haloalkyl; R¹⁶ and R¹⁷ independently represent hydrogen or any of the meanings disclosed for R¹⁵; aryl, whenever appearing in the above definitions, represents phenyl or phenyl substituted with 1, 2, 3 or 4 groups independently selected from halogen, C₁₋₄ alkyl, C₁₋₄ alkoxy, hydroxy, C₁₋₄ haloalkyl, C₁₋₄ haloalkoxy, C₁₋₄ alkylcarbonyl, C₁₋₄ alkylcarbonyloxy, C₁₋₄ alkoxycarbonyl, C₁₋₄ alkylsulfonyl, C₁₋₄ alkylsulfinyl, C₁₋₄ alkylthio, or C₁₋₄ alkylcarbonylamino; including their pharmaceutically acceptable salts and solvates.

Example embodiments of these compounds include those listed below as Group I:

1-[[1-[3-[4-(4-hydroxy-3-carboxyphenylazo) phenyl]-3methylbutanoyl]-4-piperidyl] methyl]- 1H-2-methylimidazole [4,5-c]pyridine;

trans-1-[[1-[3-[4-(4-hydroxy-3-carboxyphenylazo) phenyl] -3-phenylpropenoyl]-4-piperidyl] methyl]-1H-2-methylimidazole [4,5-c]pyridine;

1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c]pyridine

[N-[4-(4-hydroxy-3-carboxyphenylazo) benzoyl]-N-[4-(1H-2-methylimidazole [4,5-c]pyridylmethyl) phenylsulfonyl]-L-leucine ethyl ester;

[N-[4-(4-hydroxy-3-carboxyphenylazo) benzyl]-N-[4-(1H-2-methylimidazole [4,5-c]pyridylmethyl) phenylsulfonyl]-L-leucine ethyl ester;

[N-[4-(4-hydroxy-3-carboxyphenylazo) benzyl]-N-[(S)-1-isobutyl-ethoxyethyl]-N-[4-(1H-2-methylimidazole [4,5-c]pyridylmethyl) phenylsulfonamide;

cis-1-[[1-[3-[4-(4-hydroxy-3-carboxyphenylazo) phenyl]-3-phenylpropenoyl]-4-piperidyl] methyl]-1H-2-methylimidazole [4,5-c]pyridine; and

(Z)-2-hydroxy-5-[[4-[3-[4-[(2-methyl- 1H-imidazo[4,5-c]pyridin-1-yl)methyl]- 1-piperidinyl]-3-oxo-1-phenyl-1-propenyl]phenyl]azo] benzoic acid; together with their pharmaceutically acceptable salts and solvates.

The compounds used in methods of the present invention may be synthesized according to the disclosures of U.S. Pat. Nos. 5,705,504 and 5,747,477, and international published patent application numbers WO 01/77109, WO 97/09329, and W096/14317, the disclosures of which are incorporated herein by reference in their entirety.

Effect of UR-12715, 5-ASA, and the combination UR-12715 plus 5-ASA on cytokine production in vitro.

In these examples, UR-12715 and 5-ASA are delivered separately, rather than combined as the conjugate UR-12746-S, to simulate the conditions of the intestinal lumen.

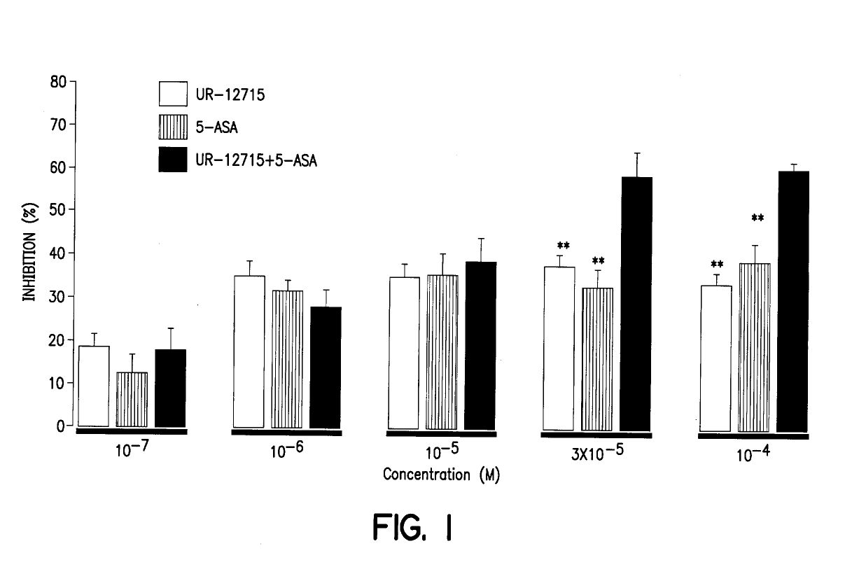

Incubation of HT-29 cells in the presence of LPS results in an increased IL-8 production in comparison with the basal release of this cytokine by the cells (15.9±0.3 vs. 1.5±0.1 ng ml⁻¹, P<0.01). When these cells are cultured in the presence of UR-12715 or 5-ASA, no significant effect is observed on IL-8 basal production (data not shown). However they show an inhibitory effect on LPS-stimulated IL-8 production, displaying a similar maximum inhibitory effect (30-40% inhibition). This is achieved at the concentration of 10⁻⁶ M without obtaining a significantly higher inhibition at increasing concentrations (up to 10⁻⁴ M) (FIG. 1). However, the combination UR-12715+5-ASA exerts a significantly higher degree of inhibition on IL-8 production at the concentrations of 3×10⁻⁵ M (58.5±11.6%, P<0.01) and 10⁻⁴ M (60.3±3.3%, P<0.01) in comparison with those obtained when both compounds are assayed separately, at equivalent concentrations (FIG. 1).

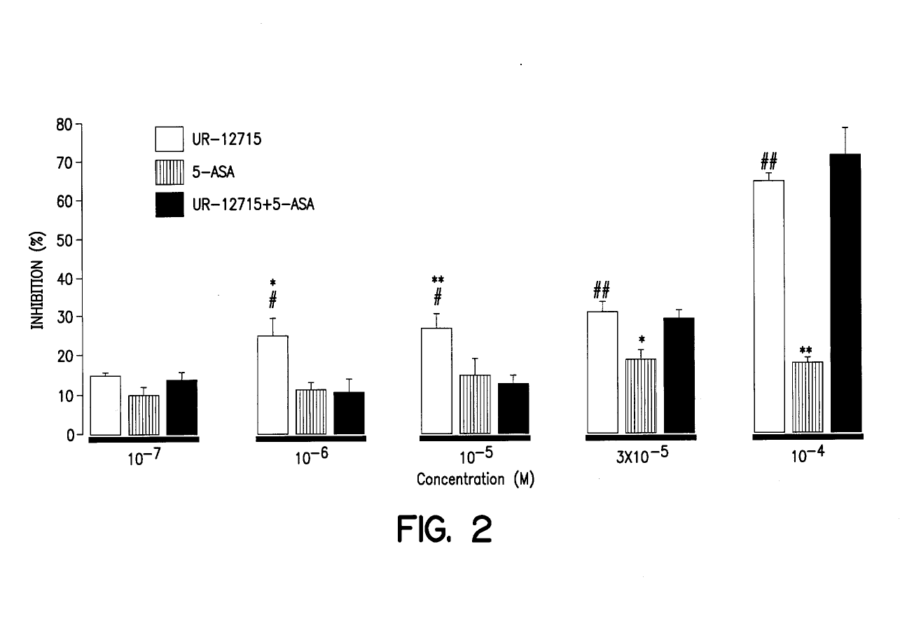

Basal IL-1β production by THP-1 cells (13.5±1.9 pg ml⁻¹) is not significantly affected by the different concentrations of the substances used in the present assay (not shown). When these cells are incubated with LPS and PMA, IL-1β production is significantly increased (1399±76pg ml⁻¹; P<0.01). Whereas 5-ASA shows a weak inhibitory effect in the different concentrations assayed (10-20% of inhibition), UR-12715 inhibits the stimulated cytokine production in a concentration-dependent way (FIG. 2). However, this inhibitory effect is not significantly enhanced after its combination with equimolar concentrations of 5-ASA.

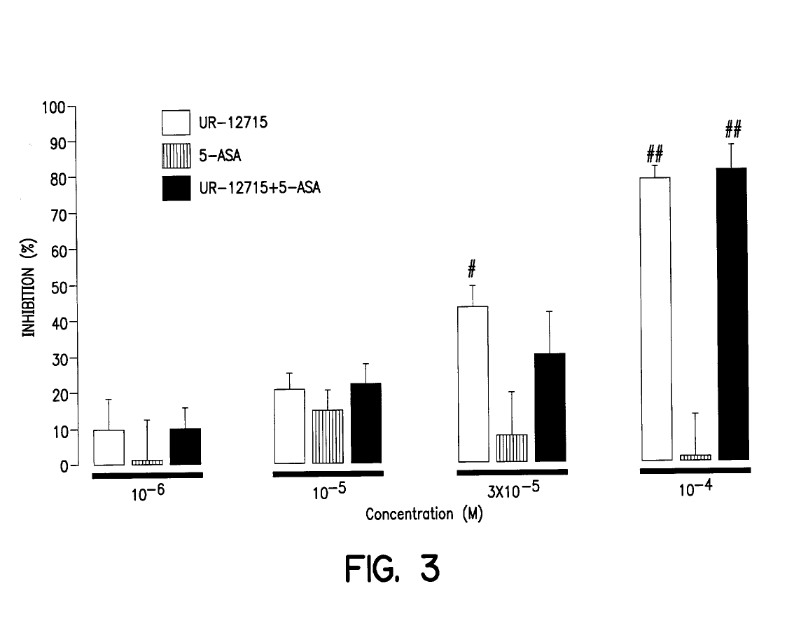

Incubation of PMA with differentiated U-937 cells in the presence of LPS for 4 h results in significantly increased TNF-α production (3863±676 pg ml⁻¹; P<0.01) over baseline (232±47 pg ml⁻¹). UR-12715 show a concentration-dependent inhibitory effect on TNF-α production after incubation of the cells with LPS, but 5-ASA is devoid of any significant effect (FIG. 3). The association of UR-12715 with 5-ASA did not result in a higher inhibition when compared with the effect show by the PAF antagonist alone.

Effect of UR-12746-S on TNBS colitic relapse/maintenance of remission

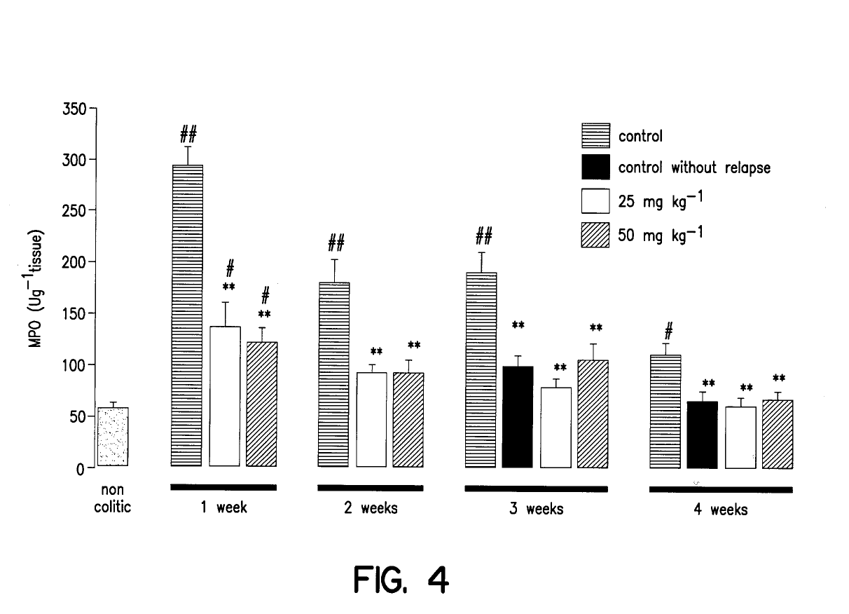

Intracolonic instillation of 10 mg of TNBS in ethanol (50% v v⁻¹) to rats results in a colonic inflammatory status with similar characteristics to those previously reported (Cruz et al., 1998). Thus one and two weeks after colitis induction, the colonic segments appeared ulcerated and inflamed, with a concomitant increase in the colonic weight/length ratio (Tables 1 and 2). MPO activity is significantly increased in comparison with non colitic animals (FIG. 4); this enzyme is a sensitive marker of neutrophil infiltration which is upregulated in experimental colitis (Krawisz et al., 1984; Yamada et al. 1992). In the present study, and in order to simulate the ‘flare-ups’ that occur in human IBD, a second intracolonic dose of 10 mg of TNBS in 50% (v v⁻¹) ethanol is administered two weeks or four weeks after the first administration. This results in a reactivation of the colonic inflammatory process in control animals when compared with the normal evolution of colonic damage in those animals which did not receive the second dose of TNBS, as evidenced by a significant increase in colonic damage score (P<0.05; Table 1) and in colonic weight/length ratio (P<0.01; Table 2). Similarly, higher values in colonic MPO activity are obtained in animals with relapse in comparison with animals without relapse (P<0.01; FIG. 4). It is conceivable to think that the reactivation of the inflammatory process obtained in the present study could be attributed, at least in part, to the ethanol vehicle used to administer TNBS. However, intracolonic administration of 0.25 ml of 50% ethanol has no impact on mucosal colonic damage when evaluated one week after its intracolonic instillation in comparison with colitic animals without relapse. Thus, the animals of this group (n=7) are assigned a median score value (range) of 4 (3-5), with a colonic weight/length ratio of 129.1±4.9 mg cm⁻¹, showing no statistical differences with the control group without relapse (P>0.1). Similarly, colonic MPO activity is not significantly enhanced one week after ethanol administration (109.5±7.3 u g vs. 98.0±10.4 u g⁻¹ in TNBS control group without relapse; P>0.1).

Pharmacological treatment of colitic rats with UR-12746-S, at the doses of 25 and 50 mg kg⁻¹, results in intestinal anti-inflammatory activity as evidenced by a reduction in colonic MPO activity during the first two weeks after the initial administration of TNBS (FIG. 4). Also, the treatment is able to prevent the increased activity in this enzyme activity after colitis relapse (FIG. 4). MPO has been widely used to detect and monitor intestinal inflammatory processes, and thus a reduction in the activity of this enzyme can be interpreted as a manifestation of the anti-inflammatory activity of a given compound (Veljaca et al., 1995). This beneficial effect of UR-12746-S is also evidenced macroscopically since it is able to attenuate the impact of the reactivation in the inflammatory process on colonic macroscopic damage score and on colonic weight/length ratio, showing no statistical differences when compared with control colitic animals without relapse (Table 1).

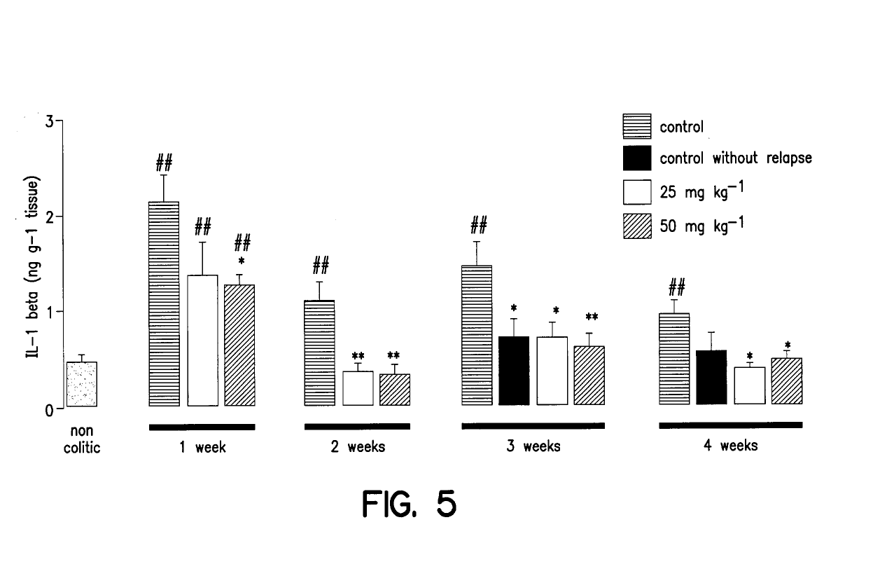

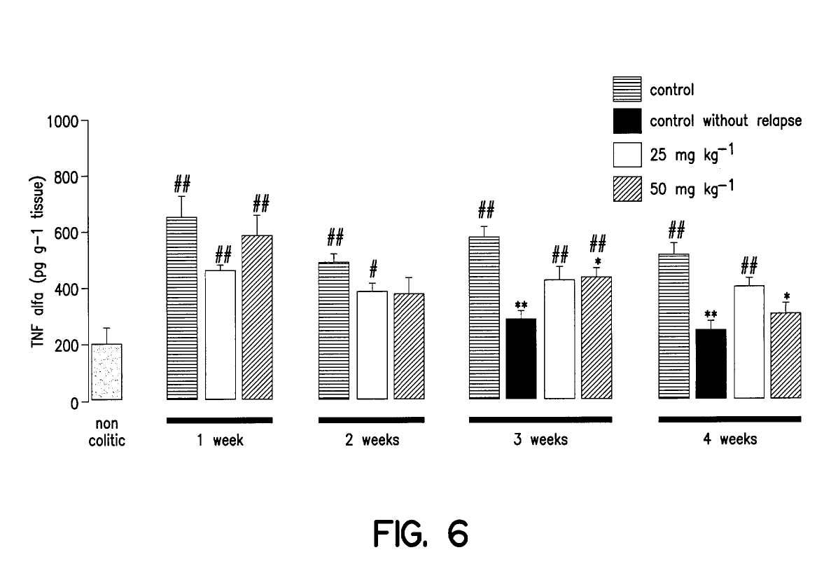

The colonic damage induced by the first administration of TNBS to rats is also characterised by a significant increase in the production of the proinflammatory cytokines IL- 1β (FIG. 5) and TNF-α (FIG. 6) compared to non colitic animals, peaking after one week and decreasing gradually over time. UR-12746-S treatment is able to decrease colonic IL-1β levels significantly during the following two weeks after the initial colonic challenge. (FIG. 5), without showing any significant effect on TNF-α levels (FIG. 6). The production of both cytokines is enhanced again after administration of the second dose of TNBS in comparison with the control animals without relapse (FIGS. 5 and 6), which levels has been normalised when compared to non colitic animals (P>0.1). Treatment with UR-12746-S effectively prevents the increase in IL-1β levels induced by the second administration of TNBS (FIG. 5) and significantly reduced, at the dose of 50 mg kg⁻¹, TNF-α levels in the inflamed tissue (FIG. 6).

When intracolonic administration of the second dose of TNBS is postponed until 4 weeks following the initial administration, induced cytokine production is reduced even further when daily doses of compounds of Formula I are administered according to the present inventive methods during the interim. These treatments effectively prevent the reoccurrence of colitic symptoms in the test animals, thus preventing relapse or, in other words, promoting maintenance of a condition of remission in the simulated chronic colitic.

The results obtained in the present study suggest that the inhibitory effect of the azo derivatives of 5-aminosalicylic acid on cytokine production also contributes their anti-inflammatory effect. Furthermore, the salts (alkali, alkaline-earth, or acid addition salts) are shown to be effective at a lower concentration than UR-12746 free acid.

IL-8 is a chemokine that actively attracts neutrophils to sites of inflammation after its secretion by inflammatory cells in response to various stimuli such as LPS, IL-1β and TNF-α. Given that epithelial cells may be the first to signal the presence of a promoter which generates the intestinal immune response, and that they contribute to IL-8 production in IBD, we have evaluated the inhibitory effects of the different test compounds on LPS stimulated IL-8 production in the HT-29 cell line, a model intestinal epithelium. The results obtained reveal that the association of UR-12715 and 5-ASA (in the case of UR-12746-S) displays a higher inhibitory effect on IL-8 production than either compound separately. The combined inhibitory effects of 5-ASA and the PAF antagonist on IL-8 production, once they are released in vivo after cleaving of the azo bond in the parent molecule by bacterial enzymes, could contribute to the amelioration in the leukocyte infiltration of the inflamed mucosa. This effect is evidenced by the decrease in colonic MPO activity exerted by UR-12746-S when assayed for its anti-inflammatory activity in vivo (FIG. 4), since MPO activity is considered as a marker of neutrophil infiltration in the inflamed mucosa (Yamada et al., 1992). In addition, the effect exerted by the simultaneous application of both compounds on this chemokine production justify the higher effect observed by UR-12746-S in reducing MPO activity in comparison with that of sulphasalazine when assayed at the same doses and in the same experimental conditions. This additive effect is now explained on the basis of the different mode of action of either the PAF antagonist UR-12715 or 5-ASA, derived from UR-12746-S, on IL-8 production and release by the cells. PAF-R antagonism may result in the disruption of the “vicious cycle” generated by IL-8 and PAF and, thus, the inhibition of the propagation of the inflammatory response.

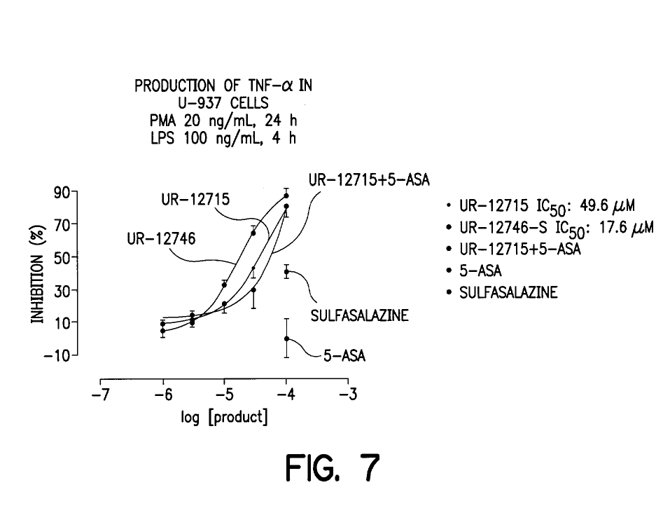

However, this additive effect is not observed when IL-1β and TNF-α production are stimulated in the two monocytic cell lines used, since only the PAF antagonist show evident inhibitory activity, while 5-ASA show either weak inhibitory activity (IL-1β) or is inactive (TNF-α) (FIG. 7). These results support the idea that UR-12715 actively participates in the anti-inflammatory activity exerts by the conjugate UR-12746-S in vivo, given the key role attributed to both IL-1β and TNF-α in the generation of the inflammatory response of the intestine PAF-R activation results in an increased production of both IL-1β and TNF-α, and thus blockade of this receptor results in down-regulation of these cytokines. This effect has been also observed to occur in vivo, since UR-12746-S administration results in a significant reduction in both cytokine levels in colitic rats.

The present study confirms the intestinal anti-inflammatory effect of UR-12746-S and demonstrates its ability to effectively ameliorate the negative consequences of relapse induced in a model of TNBS reactivated colitis. This may be of interest since the main goals in IBD therapy are to induce a remission when the disease is active and to maintain it when it is quiescent. Clearly, remission length is of outmost importance as a major determinant of disease severity and the patient's quality of life. As a consequence, when a new drug is intended to be developed for its potential use in EBD, it would be convenient to test it in an experimental model in which the relapses that characterised these intestinal conditions can be simulated.

Colitis induced in rats by the hapten TNBS has been widely used for assessing the effects of novel drugs. However, this model has some limitations given that, once TNBS has been administered intracolonically, the inflammatory status resolves spontaneously with time until complete healing of the colonic mucosa, and this is not the situation in human IBD. Other models of TNBS reactivated colitis have been previously used by other authors; in these studies, previously exposed rats are administered a second dose of the hapten either intravenously (5 mg kg⁻¹, once daily for three days) (Appleyard & Wallace, 1995) or subcutaneously (10 mg kg⁻¹, twice a day for four days) (Wallace et al., 1998) 6 weeks after the first intracolonic administration of TNBS, a time point when the initial insult has almost resolved. Recently, Bossone et al. (2001) have reported a model of reactivated colitis similar to the protocol used in the present study. Thus we promote the reactivation of the colonic inflammation by a second intracolonic administration of TNBS in the ethanol vehicle when the colonic mucosa of the rat is in process of recovery but it still shows evident signs of inflammation, both histologically (evidenced by the damage score and the elevated weight/length ratio) and biochemically (show by an increased colonic MPO activity). This is made in an attempt to better simulate the characteristics of human IBD, in which it has been described that up to 90% of patients in clinical and endoscopic remission have evidence of mucosal inflammation, including crypt abnormalities and mononuclear infiltration of the lamina propria, and even 30% show signs of acute inflammation, with neutrophil infiltration of the lamina propria, crypt abscesses and mucin depletion (Riley et al., 1991). In addition, the second dose of TNBS is administered by the intracolonic route in order to also simulate the situation in human IBD since the luminal contents of the gut are clearly important in either initiating or perpetuating the intestinal inflammatory process. The second intracolonic administration of TNBS effectively results in a reactivation of the colonic inflammatory response, as evidenced by the alteration in the different macroscopic and biochemical parameters of inflammation evaluated when compared with animals without relapse; i.e., MPO activity as well as IL- 1β and TNF-α levels.

Orally, 5-aminosalicylic acid azo derivative administration to colitic rats effectively prevents the relapse induced by a second administration of TNBS, as evidenced both macroscopically and biochemically. The inhibitory effect on colonic MPO activity and on IL-1β production is evident in all the time-points studied; i.e., before and after colitic relapse. This shows the correlation between the intestinal anti-inflammatory effect and the lower leukocyte infiltration in the inflamed mucosa, and also correlated with a decrease in IL-1β production, which has been also proposed as a marker of intestinal inflammation. In fact, this cytokine is mainly produced by mononuclear cells and thus may be considered as a more sensitive marker of inflammation than MPO activity in the chronic stages of intestinal inflammation. It is important to note that the highest dose of the drug is also able to inhibit TNF-α production, although it is only achieved after colitic relapse.

It should also be pointed out that it may be advantageous in certain embodiments of the invention to exclude from the methods of the invention the use of certain azo derivatives of 5-aminosalicylic acid, like UR-12746 free acid, or the inhibition of certain cytokines, like IL-1β. And while the azo derivatives of 5-aminosalicylic acid that are suitable for use in the invention can be administered in a wide range of dosages, for example, from about 10 mg/kg to about 10,000 mg/kg, preferably about 25 mg/kg to about 100 mg/kg (e.g., administered daily in a single dose, or optionally, in two or more divided doses), there may be situations in which a narrower range of dosages, such as about 25 mg/kg up to but not including about 50 mg/kg, might prove to be more advantageous.

### METHODS OF PREPARING THE COMPOUNDS FOR USE IN THE METHODS OF THE INVENTION

The compounds of Formula I and the species specifically disclosed for use in the methods of the present invention may be prepared, for example, according to the methods specifically disclosed in U.S. Pat. Nos. 5,705,504 and 5,747,477, and international published patent application numbers WO 01/77109, WO 97/09329, and W096/14317, respectively, the disclosures of which are hereby incorporated by reference in their entirety.

Alternatively, compounds of Formula I and the species specifically disclosed for use in the methods of the present invention can be prepared using other methods described in the literature for preparing azo bonds, for example by coupling of an amine with a nitroso compound under the reported conditions, which in general involve heating the reactants in a suitable solvent such as acetic acid.

Some compounds of the present invention can exist as different diastereoisomers and/or optical isomers. Diastereoisomers can be separated by conventional techniques such as chromatography or fractional crystallization. The optical isomers can be resolved using any of the conventional techniques of optical resolution to give optically pure isomers. Such a resolution can be performed in any chiral synthetic intermediate as well as in the products of general Formula I. Optical resolution techniques include separation by chromatography on a chiral phase or formation of a diastereoisomeric pair, resolution and subsequent recovery of the two enantiomers. The optically pure isomers can also be individually obtained using enantiospecific synthesis. The present invention covers both the individual isomers and their mixtures (e.g. racemic mixtures), whether as obtained by synthesis or by physically mixing them up.

Furthermore, some of the compounds of the present invention may exhibit cis/trans isomery. Geometric isomers can be separated by conventional techniques such as chromatography or recrystallization. Such a separation can be performed either upon the products of Formula I or upon any synthetic intermediate thereof. The individual isomers can also be obtained using stereospecific synthesis. The present invention covers each of the geometric isomers and the mixtures thereof.

### PHARMACEUTICALLY ACCEPTABLE SALTS AND SOLVATES OF COMPOUNDS FOR USE IN THE METHODS OF THE INVENTION

The compounds of Formula I and the species specifically disclosed for use in the methods of the present invention contain basic nitrogen atoms and, consequently, they can form salts with acids, which are also included in the present invention. There is no limitation on the nature of these salts, provided that, when used for therapeutic purposes, they are pharmaceutically acceptable, which, as is well-known in the art, means that they do not have reduced activity or increased toxicity compared with the free compounds. Examples of these salts include: salts with an inorganic acid such as hydrochloric acid, hydrobromic acid, hydriodic acid, nitric acid, perchloric acid, sulfuric acid or phosphoric acid; and salts with an organic acid, such as methanesulfonic acid, trifluoromethanesulfonic acid, ethanesulfonic acid, benzenesulfonic acid, p-toluenesulfonic acid, fumaric acid, oxalic acid, maleic acid, citric acid, succinic acid, tartaric acid; and other mineral and carboxylic acids well known to those skilled in the art.

The compounds of the present invention also contain a carboxy group and, consequently, they can form salts, preferably pharmaceutically acceptable salts. Examples of these salts include salts with inorganic cations such as sodium, potassium, calcium, magnesium, lithium, aluminium, zinc, etc; and salts formed with pharmaceutically acceptable amines such as ammonia, alkylamines, hydroxyalkylamines, lysine, arginine, N-methylglucamine, procaine and the like.

The salts are prepared by reacting the free compound of Formula I with a sufficient amount of the desired acid or base to produce a salt in the conventional manner. Free compounds and their salts differ in certain physicochemical properties, such as solubility in polar solvents, but they are equivalent for the purposes of the invention.

The compounds of the present invention can exist in unsolvated as well as solvated forms, including hydrated forms. In general, the solvated forms, with pharmaceutically acceptable solvents such as water, ethanol and the like, are equivalent to the unsolvated forms for the purposes of the invention.

### PHARMACEUTICAL ROUTES OF ADMINISTRATION

In accordance with the activity of the compounds herein disclosed, the present invention further provides compositions that comprise a compound of the invention together with an excipient and optionally other auxiliary agents, if necessary.

The products of the present invention will usually be administered by the oral route to mammals, including man. However, they may be adapted for other modes of administration, for example parenteral or rectal administration, the latter being the route of choice for patients with inflammatory bowel disease localized in the rectum.

Solid compositions according to the present invention for oral administration include compressed tablets, dispersible powders, granules and capsules. In tablets, the active component is admixed with at least one inert diluent such as lactose, starch, mannitol, microcrystalline cellulose or calcium phosphate; granulating and disintegrating agents for example corn starch, gelatine, microcrystalline cellulose or polyvinylpyrrolidone; and lubricating agents for example magnesium stearate, stearic acid or talc. The tablets may be coated by known techniques to delay disintegration and absorption in the gastrointestinal tract and, thereby, provide a local action at the colon. Gastric film-coated or enteric film-coated tablets can be made with sugar, gelatin, hydroxypropylcellulose, or acrylic resins. Tablets with a sustained action may also be obtained using an excipient which provides regressive osmosis, such as the galacturonic acid polymers. Formulations for oral use may also be presented as hard capsules of absorbable material, such as gelatin, wherein the active ingredient is mixed with an inert solid diluent and lubricating agents, or pasty materials, such as ethoxylated saturated glycerides. Soft gelatin capsules are also possible, wherein the active ingredient is mixed with water or an oily medium, for example coconut oil, liquid paraffin or olive oil.

Dispersible powders and granules suitable for the preparation of a suspension by the addition of water provide the active ingredient in admixture with dispersing or wetting agents, suspending agents, such as sodium carboxymethylcellulose, methylcellulose, hydroxypropylmethylcellulose, sodium alginate, polyvinylpyrrolidone, gum tragacanth, xantham gum, gum acacia, and one or more preservatives, such as methyl or propyl p-hydroxybenzoate. Additional excipients, for example sweetening, flavouring and colouring agents may also be present.

Liquid compositions for oral administration include emulsions, solutions, suspensions, syrups and elixirs containing commonly used inert diluents, such as distilled water, ethanol, sorbitol, glycerol, or propylene glycol. Such compositions may also comprise adjuvants such as wetting agents, suspending agents, sweetening, flavouring, preserving agents and buffers.

Preparations for injection, according to the present invention, for parenteral administration include sterile aqueous or non-aqueous solutions, suspensions or emulsions, in a non-toxic parentally-acceptable diluent or solvent. Examples of aqueous solvents or suspending media are distilled water for injection, Ringer's solution, and isotonic sodium chloride solution. Examples of non-aqueous solvents or suspending media are propylene glycol, polyethylene glycol, vegetable oils such as olive oil, or alcohols such as ethanol. These compositions may also include adjuvants such as wetting, preserving, emulsifying and dispersing agents. They may be sterilized by any known method or manufactured in the form of sterile solid compositions which can be dissolved in sterile water or some other sterile injectable medium immediately before use. When all of the components are sterile, the injectables will maintain sterility if they are manufactured under a sterile environment.

The products of the present invention may also be administered rectally in the form of suppositories or enemas, which include aqueous or oily solutions as well as suspensions and emulsions. Such compositions are prepared following standard procedures, well known by those skilled in the art. For example, suppositories can be prepared by mixing the active ingredient with a conventional suppository base such as cocoa butter or other glycerides.

### EXAMPLES

Example 1.

UR-12746 sodium salt (UR-12746-S) ((Z)-2-hydroxy-5-[[4-[3-[4-[(2-methyl-1H-imidazo[4,5-c]pyridin- 1-yl)methyl]-1-piperidinyl] -3 -oxo- 1 -phenyl- 1-propenyl]phenyl]azo] benzoic acid sodium salt) and UR-12715 ( (Z)-1-[4-(3-aminophenyl)-1-oxo-3-phenyl-2-propenyl]-4-[(2-methyl-lH-imidazo[4,5-c]pyridin-1-yl)methyl] piperidine) are supplied by J. Uriach and Cia S. A. (Barcelona, Spain). All other reagents, unless otherwise stated, are obtained from Sigma (St. Louis, Mo., USA). The compounds of Formula I, and particularly those selected from Group I, for use in the methods of the present invention may be prepared, for example, according to the methods specifically disclosed in U.S. Pat. Nos. 5,705,504 and 5,747,477, and international published patent application numbers WO 01/77109, WO 97/09329, and WO 96/14317, respectively, the disclosures of each of which are hereby incorporated by reference in their entirety.

Inhibition Of Cytokine Production In Cell Cultures

The human colon adenocarcinoma cell line HT-29, obtained from the Cell Culture Unit of the University of Granada (Granada, Spain) (ECACC reference number: 91072201), is used as a model of intestinal epithelium to test the ability of the different compounds to inhibit the production/release of IL-8. Cells are grown in Dulbecco's modified Eagle's medium supplemented with 10% fetal bovine serum (Boehringer Mannheim, Mannheim, Germany), 2 mM L-glutamine, 50,000 u l⁻¹ penicillin/streptomycin and 2.5 mg ml⁻¹ amphotericin B, in a humidified 5% CO₂ atmosphere at 37° C. Cells are seeded into 12-well plates and grown to confluence. Monolayers are pre-incubated for 30 min with UR- 12715 or 5-ASA or equimolar mixture of UR- 12715 plus 5-ASA, at concentrations ranging from 10⁻⁷ to 10⁻⁴ M, and then stimulated with 10 μg ml⁻¹ lipopolysaccharide (LPS). After 20 h, supernatants are collected, centrifuiged to remove debris at 7,000 g for 10 min and stored at −80° C. for the determination of IL-8 levels by enzyme-linked immunosorbent assay (Biosource International, Nivilles, Belgium).

The human monocytic cell line THP-1 is obtained from the Cell Culture Unit of the University of Granada (Granada, Spain) (ECACC reference number: 88081201) and used to assay the effects of the different compounds on IL-1 production. These cells are cultured in RPMI 1640 (Life Technologies, Paisley, UK) supplemented with 10% fetal bovine serum (Life Technologies), 2 mM glutamine and 0.05 mM mercaptoethanol in a humidified 5% CO₂ atmosphere at 37° C. Cells are seeded onto 24-well plates at a density of 2×10⁶ cells ml⁻¹ and pre-incubated during 30 min with the different test compounds, at concentrations ranging from 10⁻⁷ to 10⁻⁴ M, and then stimulated with 1 μg ml⁻¹ LPS and 1 μM phorbol 12-myristate 13-acetate (PMA). After 20 h, plates are centrifuged at 1,000 g for 10 min and supematants are collected and stored at −80° C. for the determination of IL-1 levels by EIA using a kit system (Amersham Pharmacia Biotech, Buckinghamshire, UK).

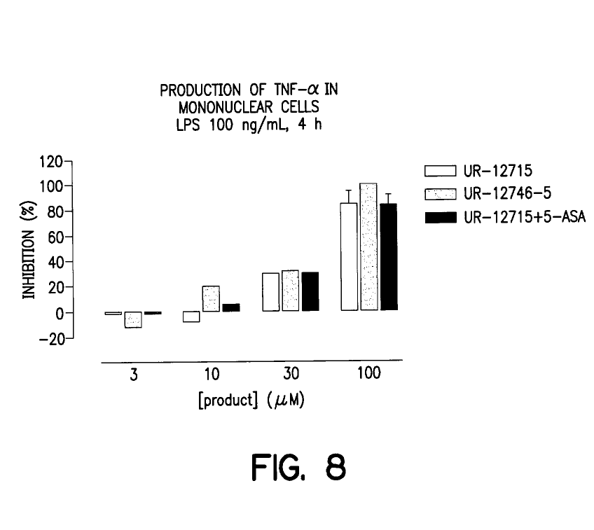

U-937 cells, a human histiocytic lymphoma cell line, are obtained from ATCC (Rockville, Md., USA) (ATCC number: CRL 1593) and used to study the effects of the different compounds on TNF-α production (FIGS. 7 and 8). The cells are cultured in RPMI 1640 (Life Technologies) supplemented with 10% fetal bovine serum and 2 mM glutamine in a humidified 5% CO₂ atmosphere at 37° C. Cell concentration is adjusted to 0.5×10⁶ cells ml, and monocytic differentiation is induced by incubation with 20 ng ml⁻¹ PMA for 24 h. After that, cells are harvested, centrifuged at 200 g for 5 min and plated onto 48-well plates at 210⁶ cells ml⁻¹, pre-incubated for 30 min with the test compounds at concentrations ranging from 10⁻⁶ to 10⁻⁴ M, and stimulated with 0.1 μg ml⁻¹ LPS. After 4 h, plates are centrifuged at 1,000 g for 10 min and supernatants are collected and stored at −80° C. for the quantification of the TNF-α released in the culture medium by EIA using a commercial kit (R&D, Minneapolis, Minn., USA).

Test compounds are prepared at 600 μM stock solution in PBS and further working dilutions are performed in culture medium. Percentage inhibition of cytokine production is calculated for every drug concentration. No cytotoxicity is detected with the studied compounds at any assayed concentration, in any cell types used, as evidenced by the trypan blue exclusion assay, which revealed a viability higher than 95% in all cases.

Induction Of Experimental Colitis

In vivo experiments are carried out in accordance with the “Guide for the Care and Use of Laboratory Animals” as promulgated by the National Institute of Health.

Colitis is induced by the method originally described by Morris et al. (1989), with minor modifications. Female Wistar rats (180-220 g) obtained from the Laboratory Animal Service of the University of Granada (Granada, Spain) are randomly distributed in several experimental groups. Animals are housed in makrolon cages (3-4 rats per cage) and maintained in an air-conditioned animal room with a 12 h light-dark cycle, and they are provided with free access to tap water and food (Panlab A.04). Animals are fasted overnight and anaesthetized with halothane. Under anesthesia, animals received 10 mg of TNBS dissolved in 0.25 ml of 50% ethanol (v v⁻¹) by means of a Teflon cannula inserted 8 cm through the anus. During and after TNBS administration rats are kept in a head-down position until they recovered from anesthesia, and then returned to their cage. A second dose of 10 mg of TNBS dissolved in 50% ethanol is administered either two or four weeks after the initial dose in an attempt to mimic the relapses common in human IBD. The animals are divided in three groups; two groups are treated orally with about 25 or 50 mg kg⁻¹ days⁻¹ of UR-12746-S suspended in 1% (w v⁻¹) methylcellulose (vol: 5 ml kg⁻¹), starting one day after the first administration of TNBS until the day before the animals are cuthanised, whereas the remaining group received vehicle (5 ml kg⁻¹1 % methylcellulose). Two additional groups are also included for reference: a non colitic group receiving intracolonically 0.25 ml of phosphate buffer saline (pH=7.4) and another colitic group which received only the first dose of TNBS (control group without relapse); both groups are orally administered with vehicle. Ten animals from each colitic group (control and UR-12746-S treated) and five from the non colitic group are sacrificed at 1, 2, 3 and 4 weeks of colitis (5 weeks in the case of the 4 week interval between TNBS administrations). Animals from the colitic control group without relapse are sacrificed at 3 and 4 weeks of colitis. Animal body weight and total food intake for each group are recorded daily.

Assessment Of Colonic Damage

Animals are sacrificed with an overdose of halothane, and the entire colon is removed. The colonic segments are placed on an ice-cold plate, cleaned of fat and mesentery, and blotted on filter paper. Each specimen is weighed and its length measured under a constant load (2 g). The colon is longitudinally opened and scored for macroscopically visible damage on a 0 to 10 scale by two observers unaware of the treatment, according to the criterion described by Bell et al. (1995), which takes into account the extension as well as the severity of colonic damage. The colon is subsequently divided longitudinally in 3 pieces for biochemical determinations. One fragment is frozen at −30° C. for myeloperoxidase (MPO) determination and the remaining samples are immediately processed for the measurement of TNF-α and IL-1β synthesis. MPO activity is measured according to the technique described by Krawisz et al. (1984); the results are expressed as MPO units per gram of wet tissue and one unit of MPO activity is defined as that degrading 1 μmol min⁻¹ of hydrogen peroxide at 25° C. Samples for the determination of TNF-α and IL-1 synthesis are immediately weighed, minced on an ice-cold plate and suspended in a tube with 10 mM sodium phosphate buffer, pH=7.4, 1:5 w v⁻¹. The tubes are placed in a shaking water bath at 37° C. for 20 min and centrifuged at 9000 g for 30 s at 4° C. The supernatants are frozen at −80° C. until assay and cytokines are quantified by enzyme-linked immunosorbent assay (Amersham Pharmacia Biotech). All biochemical measurements are completed within one week from the time of sample collection and are performed in duplicate.

Effect Of The Use ofMethods Of The Present Invention On The Spontaneous Appearance Of Colitis In HLA-B27 Transgenic Rats

HLA-B27 transgenic rat expresses HLA-B27 and human β₂-microglobulin. This transgenic animal is a suitable model for studying human inflammatory disorders. One hundred percent (100%) of these rats, by 20 weeks of age, develop chronic inflammation of the gastrointestinal tract and diarrhea. Lesions distribute throughout the stomach and intestine, with the colon being the most severe site of inflammation.

HLA-B27 female rats (derived from Fisher 344 rats, n=27) are obtained from Taconic (Germantown, N.Y., USA) at 9-11 weeks of age. Fisher 344 rats are used as non-colitic control.

HLA-B27 rats are randomized into 2 groups: Control (n=12), receiving vehicle only (1% methylcelulose, w/v); UR-12746-S (the sodium salt of UR-12746) treated (n=12), 50 mg/Kg. The compound is administered orally, by gavage, o.d., 20 mL/Kg.

A non-colitic group of Fisher rats (n=7), receiving same treatment as control will also be included.

Treatment starts as soon as animals show the first evidence of the colitis (pale stools according to a stool consistency scale, Kerr et al. (1999), J Pharmacol Exp Ther 291:903-910). In each group, 6 animals are sacrificed when control animals show important signs of colitis, such as watery diarrhea and/or bloody stools (Kerr et al., 1999). The other 6 animals of each group are allowed to extend the treatment for a period of time, depending of the progression of the diseases, in order to assess the ability of example compounds used under methods of the present invention to prevent, inhibit and/or reverse the colitic process.

At least the following parameters are measured during the treatment period: body weight (3 times a week), food consumption, and stool consistency. At least the following parameters are studied at the end of the study: colon weight, colon length, macroscopic score of colonic lesion, histopathologic analysis of colonic lesion (colon proximal, medium and distal), colonic myeloperoxidase (MPO), colonic leukotriene B₄ (LTB₄), colonic tumor necrosis factor-alpha (TNF-α), colonic interleukin 8 (IL-8), colonic nuclear factor kappa B (NF-kB, a transcription factor), colonic prostaglandins E₂ (PGE₂), colonic nitric oxide synthase (NOS), and plasmatic nitrites and nitrates.

### EXAMPLE 2

Using the same methodologies described in Example 1, trans-1-[[1-[3-[4-(4-hydroxy-3-carboxyphenylazo) phenyl] -3-phenylpropenoyl]-4-piperidyl]methyl]- 1H-2-methylimidazole [4,5-c] pyridine is tested for inhibition of cytokine production and prevention of colonic damage, and similar results are obtained. In particular, the human colon adenocarcinoma cell line HT-29 is used as a model of intestinal epithelium to test the ability of the compound trans-1-[[1-[3-[4-(4-hydroxy-3-carboxyphenylazo) phenyl]-3-phenylpropenoyl]-4-piperidyl] methyl]-1H-2-methylimidazole [4,5-c] pyridine to inhibit the production/release of MPO, LTB₄, TNF-α, IL-8, NF-KB, and PGE₂. The test compounds are prepared at 600 μM stock solution in PBS and further working dilutions are performed in culture medium. Percentage inhibition of cytokine production is calculated for every drug concentration. No cytotoxicity is detected with the studied compound at any assayed concentration, in any cell types used, as evidenced by the trypan blue exclusion assay, which revealed a viability higher than 95% in all cases.

Induction OfExperimental Colitis

Colitis is induced in Female Wistar rats (180-220 g) as described above. After receiving the second dose of 10 mg of TNBS dissolved in 50% ethanol, administered either two or four weeks after the initial dose in an attempt to mimic the relapses common in human IBD, the animals are divided in three groups; two groups are treated orally with about 25, 30, 35, 40, 45, or 50 mg kg⁻¹ day⁻¹ of trans-1-[[1-[3-[4-(4-hydroxy-3-carboxyphenylazo) phenyl]-3-phenylpropenoyl]-4-piperidyl] methyl]-1H-2-methylimidazole [4,5-c] pyridine suspended in 1% (w v⁻¹) methylcellulose (vol: 5 ml kg⁻¹), starting one day after the first administration of TNBS until the day before the animals are euthanised, whereas the remaining group received vehicle (5 ml kg⁻¹1% methylcellulose). Ten animals from each colitic group (control and trans-1-[[1-[3-[4-(4-hydroxy-3-carboxyphenylazo) phenyl]-3-phenylpropenoyl]-4-piperidyl] methyl]- 1 H-2-methylimidazole [4,5-c] pyridine treated) and five from the non colitic group are sacrificed at 1, 2, 3 and 4 weeks of colitis (5 weeks in the case of the 4 week interval between TNBS administrations). Animals from the colitic control group without relapse are sacrificed at 3 and 4 weeks of colitis. Animal body weight and total food intake for each group are recorded daily.

Assessment Of Colonic Damage

Animals are sacrificed and the colonic segments longitudinally opened and scored for macroscopically visible damage on a 0 to 10 scale by two observers unaware of the treatment, according to the criterion described by Bell et al. (1995), which takes into account the extension as well as the severity of colonic damage. A portion of the colon is subsequently treated for determination of myeloperoxidase (MPO) and the remaining samples are immediately processed for the measurement of LTB₄, TNF-α, IL-8, NF-KB, and PGE₂ synthesis.

Results

It is found that at least a majority of the parameters studied at the conclusion of the study, including at least MPO, LTB₄, TNF-α, IL-8, NF-KB, and PGE₂ levels, are inhibited by the administration of trans-1-[[1-[3-[4-(4-hydroxy-3-carboxyphenylazo) phenyl]-3-phenylpropenoyl]-4-piperidyl] methyl]-1H-2-methylimidazole [4,5-c] pyridine by the methods of the present invention.

In conclusion, the results obtained in the present study support the use of trans-1-[[1-[3-[4-(4-hydroxy-3-carboxyphenylazo) phenyl]-3-phenylpropenoyl]-4-piperidyl] methyl]-1H-2-methylimidazole [4,5-c] pyridineagainst intestinal inflammation, in view of their ability both to facilitate the recovery of the inflamed mucosa or prevent damage to the mucosa, to ameliorate the impact in the reactivation of the inflammatory process, and to prevent recurrence of such reactivation or relapse. Inhibition of proinflammatory cytokines, as evidenced both in vitro and in vivo in the present study, offers an opportunity to disrupt the inflammatory cascade at an early stage, inhibiting subsequent recruitment and activation of immunoregulatory cells and their release of inflammatory mediators.

### EXAMPLE 3

Using the same methodologies described in Examples 1 and 2, 1-[[I-[[N-[[4-(4-hydroxy-3-carboxyphenylazo) phenyl] sulfonyl]-N-phenylamino] acetyl]-4-piperidyl] methyl]-1H-2-methylimidazole [4,5-c] pyridine is tested for inhibition of cytokine production and prevention of colonic damage, and similar results are obtained.

### EXAMPLE 4

Using the same methodologies described in Examples 1 and 2, [N-[4-(4-hydroxy-3-carboxyphenylazo) benzoyl]-N-[4-(1H-2-methylimidazole [4,5-c] pyridylmethyl) phenylsulfonyl]-L-leucine ethyl ester is tested for inhibition of cytokine production and prevention of colonic damage, and similar results are obtained.

### EXAMPLE 5

Using the same methodologies described in Examples 1 and 2, [N-[4-(4-hydroxy-3-carboxyphenylazo) benzyl]-N-[4-(1H-2-methylimidazole [4,5-c] pyridylmethyl) phenylsulfonyl]-L-leucine ethyl ester is tested for inhibition of cytokine production and prevention of colonic damage, and similar results are obtained.

### EXAMPLE 6

Using the same methodologies described in Examples 1 and 2, [N-[4-(4-hydroxy-3-carboxyphenylazo) benzyl]-N-[(S)- 1-isobutyl-ethoxyethyl]-N-[4-(1H-2-methylimidazole [4,5-c] pyridylmethyl) phenylsulfonamide is tested for inhibition of cytokine production and prevention of colonic damage, and similar results are obtained.

Statistical Analysis

All results are expressed as mean ± s.e.m. Differences among means are tested for statistical significance using one way analysis of variance (ANOVA) and post hoc least significance tests. Non-parametric data (score) are expressed as median (range) and are analysed with the Mann-Whitney U test. Statistical significance is set at P<0.05. In chronic experiments, data from non-colitic animals, which did not differ significantly from one another, are pooled together and presented as a single group.

Results

It is found that at least a majority of the parameters studied at the conclusion of the study, including at least MPO, LTB₄, TNF-α, IL-8, NF-RB, and PGE₂ levels, are inhibited by the administration of the above-mentioned compounds by the methods of the present invention.

In conclusion, the results obtained in the present study support the use of salts of azo derivatives of 5-aminosalicylic acid against intestinal inflammation, in view of their ability both to facilitate the recovery of the inflamed mucosa or prevent damage to the mucosa, to ameliorate the impact in the reactivation of the inflammatory process, and to prevent recurrence of such reactivation or relapse. Inhibition of proinflammatory cytokines, as evidenced both in vitro and in vivo in the present study, offers an opportunity to disrupt the inflammatory cascade at an early stage, inhibiting subsequent recruitment and activation of immunoregulatory cells and their release of inflammatory mediators.

TABLE 1
Effects of UR-12746-S treatment (25 and 50 mg kg⁻¹) on damage
score (0-10) in reactivated TNBS colitis.

| | 1 wk | 2 wk | 3 wk | 4 wk |
|---|---|---|---|---|
| Non colitic | 0 | | | |
| TNBS Control: | | | | |
| with relapse | 5.5 (4-6) | 4.5 (4-6) | 6 (5-7) | 6 (4-7) |
| without relapse | | | 3.5 (3-5)* | 3 (2-5)* |
| UR-12746-S: | | | | |
| 25 mg kg⁻¹ | 5.5 (4-6) | 5 (4-5) | 5 (4-6) | 5 (2-6) |
| 50 mg kg⁻¹ | 4.5 (4-6) | 4 (3-5) | 4 (3-5)* | 3 (2-4)* |

Score data are expressed as median (range).
P < 0.05 vs. TNBS control group with relapse.
All groups differ significantly from the non-colitic group (P < 0.01, not shown).

TABLE 2
Effects of UR-12746-S treatment (25 and 50 mg kg⁻¹) on colonic
weight/length ratio (mg cm⁻¹) in reactivated TNBS colitis.

| | 1 wk | 2 wk | 3 wk | 4 wk |
|---|---|---|---|---|
| Non colitic | 86.0 ± 2.1 | | | |
| TNBS Control: | | | | |
| with relapse | 152.5 ± 5.2 | 140.5 ± 4.9 | 161.4 ± 6.3 | 152.4 ± |
| | | | | 9.2 |
| without relapse | | | 121.4 ± 6.8** | 104.0 ± |
| | | | | 4.5** |
| UR-12746-S. | | | | |
| 25 mg kg⁻¹ | 146.6 ± 8.1 | 143.6 ± 4.9 | 150.5 ± 4.3 | 143.2 ± |
| | | | | 8.1 |
| 50 mg kg⁻¹ | 137.0 ± 8.4 | 120.0 ± 5.9* | 142.3 ± 3.1* | 110.8 ± |
| | | | | 3.7** |

Data are expressed as mean ± s.e.mean.
*P < 0.05,
**P < 0.01 vs TNBS control group.
All groups differ significantly from the non-colitic group (P < 0.01, not shown).

### BIBLIOGRAPHY

BELL, C. J., GALL, D. G. & WALLACE, J. L. (1995). Disruption of colonic electrolyte transport in experimental colitis. Am. J Physiol., 268, G622-G630.

BOSSONE, C., HOSSEINI, J. M., PINEIRO-CARRERO, V. & SHEA-DONOHUE, T. (2001). Alteration in spontaneous contractions in vitro after repeated inflammation of rat distal colon. Am. J Physiol., 280, G949-G957.

FIOCCHI, C. (1998). Inflammatory bowel disease: etiology and pathogenesis. Gastroenterology, 115, 182-205.

GALVEZ, J., GARRIDO, M., MERLOS, M., TORRES, M. I. & ZARZUELO, A. (2000). Intestinal anti-inflammatory activity of UR- 12746-S, a novel 5-ASA conjugate, on acute and chronic experimental colitis in the rat. Br. J Pharmacol., 130, 1949-1959.

KATZ, J. A., ITOH, J. & FIOCCHI, C. (1999). Pathogenesis of inflammatory bowel disease. Curr. Opin. Gastroenterol, 15, 291-297.

KHO, Y. H., POOL, M. O., JANSMAN, F. G. & HARTING, J. W. (2001). Pharmacotherapeutic options in inflammatory bowel disease: an update. Pharm. World. Sci., 23, 17-21.

KRAWISZ, J. E., SHARON, P. & STENSON, W. F. (1984). Quantitative assay for acute intestinal inflammation based on myeloperoxidase activity. Assessment of inflammation in rat and hamster models. Gastroenterology, 87, 1344-1350.

MAKINS, R. J. & COWAN, R. E. (2001). 5-amino-salicylate in the management of inflammatory bowel disease. Colorectal Dis., 3, 218-222.

MEENAN, J., GROOL, T. A., HOMMES, D. W., DIJKHUIZEN, S., TEN KATE, F. J.,

WOOD, M., WHITTAKER, M., TYTGAT, G. N. & VAN DEVENTER S. J. (1996). Lexipafant (BB-882). a platelet activating antagonist, ameliorates mucosal inflammation in an animal model of colitis. Eur. J Gastroenterol. Hepatol., 8, 569-583.

MORRIS, G. P., BECK, P. L. HERRIDGE, W., DEPEW, W., SZEWCZUK, M. R. & WALLACE J. L. (1989). Hapten-induced model of chronic inflammation and ulceration in the rat colon. Gastroenterology, 96, 795-803.

PODOLSKY, D. K. & FIOCCHI, C. (2000) Cytokines, chemokines, growth factors, eicosanoids and other bioactive molecules in inflammatory bowel disease. In Inflammatory Bowel Disease, 5^{th} Ed. ed. Kirsner, J. B. pp. 191-207. Philadelphia: W. B. Saunders.

RILEY, S. A., MANI, V., GOODMAN, M. J., DUTT, S. & HERD, M. E. (1991). Microscopic actitivity in ulcerative colitis: what does it mean? Gut, 32, 174-8.

TRAVIS S. P. L. & JEWELL, D. P. (1994). Salicylates for ulcerative colitis-their mode of action. Pharmacol Ther., 63, 135-161.

VELJACA, M., LESCH, C. A., PLLANA, R., SANCHEZ, B., CAHN, K. & GUGLIETTA, A. (1995). BPC-15 reduces trinitrobenzene sulfonic-induced colonic damage in rats. J. Pharmacol. Exp. Ther., 272,417-422.

WALLACE, J. L. (1988). Release of platelet-activating factor (PAF) and accelerated healing induced by a PAF antagonist in an animal model of chronic colitis. Can. J Physiol. Pharmacol., 66, 422-425.

YAMADA, T., MARSHALL, S., SPECIAN, R. D. & GRISHAM, M. B. (1992). A comparative study of two models of experimental colitis in rats. Gastroenterology, 102, 1524-1534.

What is claimed is:

1. A method of ameliorating negative effects of relapse of a inflammatory bowel disease in a mammal comprising administering to a mammal having suffered from inflammatory bowel disease an effective amount of a compound of Formula I: 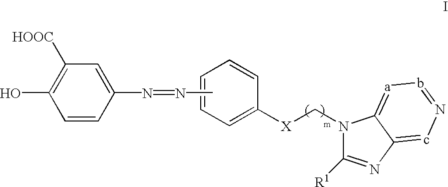
wherein:
the 4-hydroxy-3-carboxyphenylazo moiety can be at the 3- or 4-position of the benzene ring;
m represents 1 or 2;
R¹ represents C₁₋₄ alkyl or C₃₋₇ cycloalkyl;
a, b and c represent CR², wherein each R² independently represents hydrogen or C₁₋₄alkyl;
X represents a group of formula (i) or (ii): 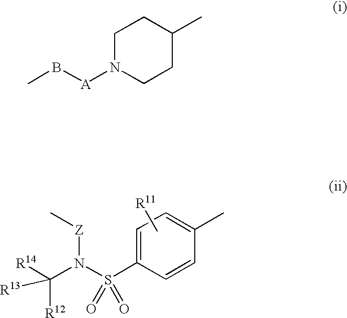
wherein these groups are bound to the phenyl ring in formula I via B and Z, respectively;
A represents -CO-, -SO₂-, -NHCO- or -OCO-;
B represents a group of formula (iii), and when A represents -CO- or -SO₂-, B can also represent a group of formula (iv), (v), (vi) or (vii); 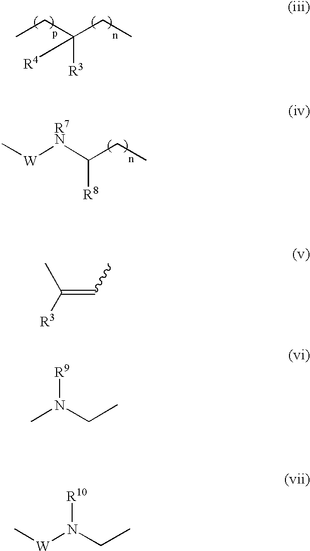
n represents 0, 1, 2 or 3;
p represents O or I;
R³represents hydrogen, C₁₋₄ alkyl, C₁₋₄ haloalkyl, C₃₋₇ cycloalkyl, C₁₄ alkoxy-C₁₋₄ alkyl or aryl;
R⁴ represents hydrogen, C₁₋₄ alkyl, -COOR or -CONR⁵R⁶, and when A represents -CO- or -SQ-, then R⁴ can also represent -NR⁵R⁶, -NR⁷C(=O)OR⁵, -NR⁷C(=O)R⁵, -NR⁷C(=O)NR⁵R⁶ or -NR7SO₂R⁵; or R³ and R⁴ together form a C₂₋₆ polyrnethylene chain;
R⁵represents C₁₋₄ alkyl, aryl or aryl-C₁₋₄ alkyl;
R⁶ and R⁷ independently represent hydrogen or C₁₋₄ alkyl;
W represents -OC(=O)-, -C(=O)-, -NR⁶C(=O)- or -SO₂-;
R⁸represents aryl;
R9 represents C₁₋₄ alkyl, C₃₋₇ cycloalkyl, -C(=O)OR⁵, -C(=O)R⁵, -C(O)NR⁵R⁶, or-SO2R⁵;
R¹⁰ represents C₁₋₄ alkyl, C₃₋₇ cycloalkyl, aryl, or aryl-C₁₋₄ alkyl;
Z represents (CH2)_{q}CO- or-(CH₂)ᵣ-q represents 0, 1 or 2;
r represents 1 or 2;
R¹¹ represents hydrogen or halogen;
R¹² and R¹³ independently represent hydrogen, C₁₋₆ alkyl, C₃₋₇ cycloalkyl or C₃₋₇ cycloalkyl-C₁₋₆ alkyl;
or R¹² and R¹³ together form a C₂₋₆ polyrnethylene chain;
R¹⁴ represents -COR¹⁵, -COOH, -COOR¹⁵, -CONR¹⁶R¹⁷, -C₁₋₆ alkyl-OR¹⁵, -C₁₋₆ alkyl-OC(=O)R¹⁵ or -C₁₋₆ alkyl-OC(=O)NR¹⁶R¹⁷;
R¹⁵ represents C₁₋₆ alkyl, C₂₋₆ alkenyl, C₂₆alkynyl, C₃-₇ cycloalkyl or C₁₋₆ haloalkyl;
R¹⁶ and R¹⁷ independently represent hydrogen or any of the meanings disclosed for R¹⁵;
aryl, whenever appearing in the above definitions, represents phenyl or phenyl substituted with 1, 2, 3 or 4 groups independently selected from halogen, C₁₄ alkyl, C₁₋₄alkoxy, hydroxy, C₁₋₄haloalkyl, C₁₋₄haloalkoxy, C₁₄ alkylcarbonyl, C₁₋₄alkylcarbonyloxy, C₁₋₄alkoxycarbonyl, C₁₋₄alkylsulfonyl, C₁₋₄alkylsulfinyl, C₁₋₄alkylthio, or C₁₋₄alkylcarbonylamino; and
the pharmaceutically acceptable salts and solvates thereof.

2. The method of claim 1 wherein said compound is 1-[[1-[3-[4-(4-hydroxy-3-carboxyphenylazo) phenyl]-3methylbutanoyl]-4-piperidyl]methyl]- 1H-2-methylimidazole [4,5-c]pyridine sodium salt or pharmaceutically acceptable solvates thereof.

3. The method of claim 1 wherein said compound is trans-1-[[1-[3-[4-(4-hydroxy-3-carboxyphenylazo) phenyl]-3-phenylpropenoyl]-4-piperidyl] methyl]-1H-2-methylimidazole [4,5-c]pyridine sodium salt or pharmaceutically acceptable solvates thereof.

4. The method of claim 1 wherein said compound is 1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c]pyridine sodium salt or pharmaceutically acceptable solvates thereof.

5. The method of claim 1 wherein said compound is [N-[4-(4-hydroxy-3-carboxyphenylazo) benzoyl]-N-[4-(1H-2-methylimidazole [4,5-c]pyridylmethyl) phenylsulfonyl]-L-leucine ethyl ester sodium salt or pharmaceutically acceptable solvates thereof.

6. The method of claim 1 wherein said compound is [N-[4-(4-hydroxy-3-carboxyphenylazo) benzyl]-N-[4-( 1H-2-methylimidazole [4,5 -c]pyridylmethyl) phenylsulfonyl]-L-leucine ethyl ester sodium salt or pharmaceutically acceptable solvates thereof.

7. The method of claim 1 wherein said compound is [N-[4-(4-hydroxy-3-carboxyphenylazo) benzyl]-N-[(S)- 1-isobutyl-ethoxyethyl]-N-[4-(1H-2-methylimidazole [4,5-c]pyridylmethyl) phenylsulfonamide sodium salt or pharmaceutically acceptable solvates thereof.

8. The method of claim 1 wherein said compound is cis-1-[[1-[3-[4-(4-hydroxy-3-carboxyphenylazo) phenyl]-3-phenylpropenoyl]-4-piperidyl] methyl]-1H-2
-methylimidazole [4,5-c]pyridine sodium salt or pharmaceutically acceptable solvates thereof.

9. The method of claim 1 wherein said compound is (Z)-2-hydroxy-5-[[4-[3-[4-[(2-methyl-1H-imidazo[4,5-c]pyridin- 1-yl)methyl]-1-piperidinyl]-3-oxo-1-phenyl-1-propenyl]phenyl]azo] benzoic acid sodium salt or pharmaceutically acceptable solvates thereof.

10. The method of claim 1 wherein said inflammatory bowel disease comprises one or more of chronic gastrointestinal inflammation, colitis, ulcerative colitis or Crohn's disease.

11. The method of claim 1 wherein said effective amount falls in the range of about 10 mg/kg to about 10,000 mg/kg.

12. The method of claim 1 wherein said effective amount falls in the range of about 25 mg/kg to about 100 mg/kg.

13. The method of claim 1 wherein said effective amount is administered at least daily, optionally in two or more divided doses.

14. The method of claim 1 wherein said effective amount is administered daily for a period of at least 7 days.

15. The method of claim 1 wherein said effective amount is administered daily for a period of at least 2 weeks.

16. The method of claim 1 wherein said effective amount is administered daily for a period of at least 4 weeks.

17. The method of claim 1 wherein said effective amount is delivered orally.

18. A method of preventing a relapse of inflammatory bowel disease in a mammal comprising administering to a mammal having suffered from inflammatory bowel disease an effective amount of a compound of Formula I: 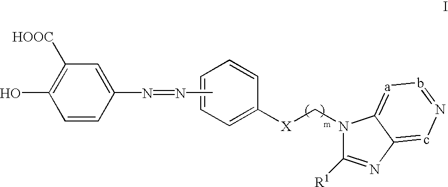
wherein: the 4-hydroxy-3-carboxyphenylazo moiety can be at the 3- or 4-position of the benzene ring;
m represents 1 or 2;
R¹ represents C₁₋₄alkyl or C₃₋₇cycloalkyl;
a, b and c represent CR², wherein each R² independently represents hydrogen or C₁₋₄alkyl;
X represents a group of formula (i) or (ii): 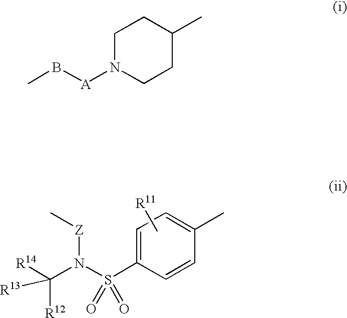
wherein these groups are bound to the phenyl ring in formula I via B and Z, respectively;
A represents -CO-, -SO₂-, -NHCO- or -OCO-;
B represents a group of formula (iii), and when A represents -CO- or -SO₂-, B can also represent a group of formula (iv), (v), (vi) or (vii); 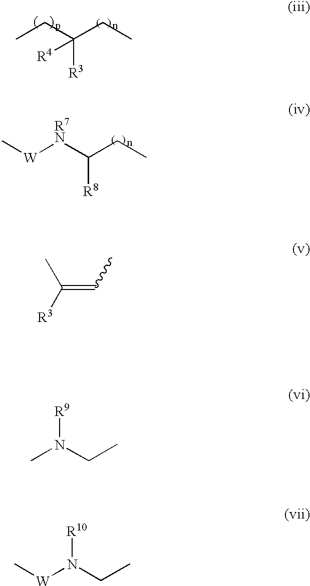
n represents 0, 1, 2 or 3;
p represents O or I;
R³represents hydrogen, C₁₋₄ alkyl, C₁₋₄ haloalkyl, C₃₋₇ cycloalkyl, C₁₋₄ alkoxy-C₁₋₄ alkyl or aryl;
R⁴ represents hydrogen, C₁₋₄ alkyl, -COOR⁵ or -CONR⁵R⁶, and when A represents -CO- or -SQ-, then R⁴ can also represent -NR⁵R⁶, -NR⁷C(=O)OR⁵, -NR⁷C(=O)R⁵, -NR⁷C(=O)NR⁵R⁶ or NR⁷SO₂R⁵ or R³ and R⁴ together form a C₂₋₆polymrethylene chain;
R⁵represents C₁₋₄ alkyl, aryl or aryl-C₁₋₄ alkyl;
R⁶ and R⁷ independently represent hydrogen or C₁₋₄ alkyl;
W represents -OC(=O)-, -C(=O)-, -NR⁶C(=O)- or -SO₂-;
R⁸ represents aryl;
R9 represents C₁₋₄ alkyl, C₃₋₇ cycloalkyl, -C(=O)OR⁵, -C(=O)R⁵, -C(=O)NR⁵R⁶, or -SO2R⁵;
R¹⁰ represents C₁₋₄ alkyl, C₃₋₇ cycloalkyl, aryl, or aryl-C₁₋₄alkyl;
Z represents (CH₂)_{q}CO- or -(CH₂)ᵣ-q represents 0, 1 or 2;
r represents 1 or 2;
R¹¹ represents hydrogen or halogen;
R¹² and R¹³ independently represent hydrogen, C₁₋₆ alkyl, C₃₋₇ cycloalkyl or C₃₋₇ cycloalkyl-C₁₋₆ alkyl;
or R¹² and R¹³ together form a C₂₋₆ polyrnethylene chain;
R¹⁴ represents -COR , -COOH, -COOR¹⁵, -CONR¹⁶R¹⁷, -C₁₋₆ alkyl-OR¹⁵, -C₁₋₆ alkyl-OC(=O)R¹⁵ or -C₁₋₆alkyl-OC(=O)NR¹⁶R¹⁷;
R¹⁵ represents C₁₋₆ alkyl, C₂₋₆ alkenyl, C₂₆alkynyl, C₃₋₇ cycloalkyl or C₁₋₆ haloalkyl;
R¹⁶ and R¹⁷ independently represent hydrogen or any of the meanings disclosed for R¹⁵;
aryl, whenever appearing in the above definitions, represents phenyl or phenyl substituted with 1, 2, 3 or 4 groups independently selected from halogen, C₁₋₄ alkyl, C₁₋₄ alkoxy, hydroxy, C₁₋₄ haloalkyl, C₁₋₄ haloalkoxy, C₁₋₄ alkylcarbonyl, C₁₋₄ alkylcarbonyloxy, C₁₋₄ alkoxycarbonyl, C₁₋₄ alkylsulfonyl, C₁₋₄ alkylsulfinyl, C₁₋₄ alkylthio, or C₁₋₄ alkylcarbonylamino; and
the pharmaceutically acceptable salts and solvates thereof.

19. The method of claim 18 wherein said compound is 1-[[1-[3-[4-(4-hydroxy-3-carboxyphenylazo) phenyl]-3methylbutanoyl]-4-piperidyl] methyl]- 1H-2-methylimidazole [4,5-c]pyridine sodium salt or pharmaceutically acceptable solvates thereof.

20. The method of claim 18 wherein said compound is trans-1-[[1-[3-[4-(4-hydroxy-3-carboxyphenylazo) phenyl]-3-phenylpropenoyl]-4-piperidyl] methyl]-1H-2-methylimidazole [4,5-c]pyridine sodium salt or pharmaceutically acceptable solvates thereof.

21. The method of claim 18 wherein said compound is 1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c]pyridine sodium salt or pharmaceutically acceptable solvates thereof.

22. The method of claim 18 wherein said compound is [N-[4-(4-hydroxy-3-carboxyphenylazo) benzoyl]-N-[4-(1H-2-methylimidazole [4,5-c]pyridylmethyl) phenylsulfonyl]-L-leucine ethyl ester sodium salt or pharmaceutically acceptable solvates thereof.

23. The method of claim 18 wherein said compound is [N-[4-(4-hydroxy-3-carboxyphenylazo) benzyl]-N-[4-(1H-2-methylimidazole [4,5-c]pyridylmethyl) phenylsulfonyl]-L-leucine ethyl ester sodium salt or pharmaceutically acceptable solvates thereof.

24. The method of claim 18 wherein said compound is [N-[4-(4-hydroxy-3-carboxyphenylazo) benzyl]-N-[(S)- 1-isobutyl-ethoxyethyl]-N-[4-(1H-2-methylimidazole [4,5-c]pyridylmethyl) phenylsulfonamide sodium salt or pharmaceutically acceptable solvates thereof.

25. The method of claim 18 wherein said compound is cis-1-[[1-[3-[4-(4-hydroxy-3-carboxyphenylazo) phenyl]-3-phenylpropenoyl]-4-piperidyl] methyl]-1H-2-methylimidazole [4,5-c]pyridine sodium salt or pharmaceutically acceptable solvates thereof.

26. The method of claim 18 wherein said compound is (Z)-2-hydroxy-5-[[4-[3-[4-[(2-methyl-1H-imidazo[4,5-c]pyridin- 1 -yl)methyl] -1-piperidinyl]-3-oxo-1-phenyl- 1-propenyl]phenyl]azo] benzoic acid sodium salt or pharmaceutically acceptable solvates thereof.

27. The method of claim 18 wherein said inflammatory bowel disease comprises one or more of chronic gastrointestinal inflammation, colitis, ulcerative colitis or Crohn's disease.

28. The method of claim 18 wherein said effective amount falls in the range of about 10 mg/kg to about 10,000 mg/kg.

29. The method of claim 18 wherein said effective amount falls in the range of about 25 mg/kg to about 100 mg/kg.

30. The method of claim 18 wherein said effective amount is administered at least daily, optionally in two or more divided doses.

31. The method of claim 18 wherein said effective amount is administered daily for a period of at least 7 days.

32. The method of claim 18 wherein said effective amount is administered daily for a period of at least 2 weeks.

33. The method of claim 18 wherein said effective amount is administered daily for a period of at least 4 weeks.

34. The method of claim 18 wherein said effective amount is delivered orally.

35. A method of inhibiting production of one or more cytokines in a mammal comprising administering to a mammal in need thereof an effective amount of a compound selected from the group consisting of pharmaceutically acceptable salts of:
1-[[1-[3-[4-(4-hydroxy-3-carboxyphenylazo) phenyl]-3methylbutanoyl]-4-piperidyl] methyl]-1H-2-methylimidazole [4,5-c]pyridine;
trans-1-[[1-[3-[4-(4-hydroxy-3-carboxyphenylazo) phenyl]-3-phenylpropenoyl]-4-piperidyl] methyl]-1H-2-methylimidazole [4,5-c]pyridine;
1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c]pyridine [N-[4-(4-hydroxy-3-carboxyphenylazo) benzoyl]-N-[4-(1H-2-methylimidazole [4,5-c]pyridylmethyl) phenylsulfonyl]-L-leucine ethyl ester;
[N-[4-(4-hydroxy-3-carboxyphenylazo) benzyl]-N-[4-(1H-2-methylimidazole [4,5-c]pyridylmethyl) phenylsulfonyl]-L-leucine ethyl ester;
[N-[4-(4-hydroxy-3-carboxyphenylazo) benzyl]-N-[(S)-1-isobutyl-ethoxyethyl]-N-[4-(1H-2-mcthylimidazole [4,5-c]pyridylmethyl) phenylsulfonamide;
cis- 1-[[1-[3-[4-(4-hydroxy-3-carboxyphenylazo) phenyl]-3-phenylpropenoyl]-4-piperidyl] methyl]-1H-2-methylimidazole [4,5-c]pyridine; and
(Z)-2-hydroxy-5-[[4-[3-[4-[(2-methyl- 1H-imidazo[4,5-c]pyridin- 1-yl)methyl]-1-piperidinyl]-3-oxo-1-phenyl-1-propenyl]phenyl]azo] benzoic acid; including pharmaceutically acceptable solvates thereof.

36. The method of claim 35 wherein said compound is 1-[[1-[3-[4-(4-hydroxy-3-carboxyphenylazo) phenyl]-3methylbutanoyl]-4-piperidyl] methyl]-1H-2-methylimidazole [4,5-c]pyridine sodium salt or pharmaceutically acceptable solvates thereof.

37. The method of claim 35 wherein said compound is trans-1-[[l-[3-[4-(4-hydroxy-3-carboxyphenylazo) phenyl]-3-phenylpropenoyl]-4-piperidyl] methyl]-1H-2-methylimidazole [4,5-c]pyridine sodium salt or pharmaceutically acceptable solvates thereof.

38. The method of claim 35 wherein said compound is 1-[[1-[[N-[[4-(4-hydroxy-3-carboxyphenylazo) phenyl] sulfonil]-N-phenylamino] acetyl]-4-piperidyl] methyl]-1H-2-methylimidazole [4,5-c]pyridine sodium salt or pharmaceutically acceptable solvates thereof.

39. The method of claim 35 wherein said compound is [N-[4-(4-hydroxy-3-carboxyphenylazo) benzoyl]-N-[4-(1H-2-methylimidazole [4,5-c]pyridylmethyl) phenylsulfonyl]-L-leucine ethyl ester sodium salt or pharmaceutically acceptable solvates thereof.

40. The method of claim 35 wherein said compound is [N-[4-(4-hydroxy-3-carboxyphenylazo) benzyl]-N-[4-(1H-2-methylimidazole [4,5-c]pyridylmethyl) phenylsulfonyl]-L-leucine ethyl ester sodium salt or pharmaceutically acceptable solvates thereof.

41. The method of claim 35 wherein said compound is [N-[4-(4-hydroxy-3-carboxyphenylazo) benzyl]-N-[(S)-1-isobutyl-ethoxyethyl]-N-[4-(1H-2-methylimidazole [4,5-c]pyridylmethyl) phenylsulfonamide sodium salt or pharmaceutically acceptable solvates thereof.

42. The method of claim 35 wherein said compound is cis-1-[[1-[3-[4-(4-hydroxy-3-carboxyphenylazo) phenyl]-3-phenylpropenoyl]-4-piperidyl] methyl]- 1H-2-methylimidazole [4,5-c]pyridine sodium salt or pharmaceutically acceptable solvates thereof.

43. The method of claim 35 wherein said compound is (Z)-2-hydroxy-5-[[4-[3-[4-[(2-methyl- 1H-imidazo[4,5-c]pyridin- 1-yl)methyl]- 1 -piperidinyl]-3-oxo- 1-phenyl-1-propenyl]phenyl]azo] benzoic acid sodium salt or pharmaceutically acceptable solvates thereof.

44. The method of claim 35 wherein said one or more cytokines includes interleukin-8 or tumor necrosis factor-α.

45. The method of claim 35 wherein said production of one or more cytokines is caused by inflammatory bowel disease, including one or more of chronic gastrointestinal inflammation, colitis, ulcerative colitis or Crohn's disease.

46. The method of claim 35 wherein said effective amount falls in the range of about 10 mg/kg to about 10,000 mg/kg.

47. The method of claim 35 wherein said effective amount falls in the range of about 25 mg/kg to about 100 mg/kg.

48. The method of claim 35 wherein said effective amount is administered at least daily, optionally in two or more divided doses.

49. The method of claim 35 wherein said effective amount is administered daily for a period of at least 7 days.

50. The method of claim 35 wherein said effective amount is administered daily for a period of at least 2 weeks.

51. The method of claim 35 wherein said effective amount is administered daily for a period of at least 4 weeks.

52. The method of claim 35 wherein said effective amount is delivered orally.
